(19) United States
(12) Patent Application Publication
(10) Pub. No.: US 2025/0025229 A1
(43) Pub. Date: Jan. 23, 2025

(54) RENAL NEUROMODULATION

(71) Applicant: THE REGENTS OF THE UNIVERSITY OF CALIFORNIA, Oakland, CA (US)

(72) Inventors: Olujimi A. Ajijola, Sherman Oaks, CA (US); Yuichi Hori, Los Angeles, CA (US)

(21) Appl. No.: 18/264,701

(22) PCT Filed: Feb. 8, 2022
(86) PCT No.: PCT/US22/15696

§ 371 Date: Aug. 8, 2023

Related U.S. Application Data
(60) Provisional application No. 63/146,962, filed on Feb. 8, 2021.

(51) Int. Cl. A61B 18/14; A61B 18/00

(52) U.S. Cl. CPC A61B 18/1492; A61B 2018/00511; A61B 2218/002

(57) ABSTRACT

The present invention provides methods for renal denervation for the effective treatment of systemic adrenergic activation and related diseases and disorders, including but not limited to cardiovascular diseases. In certain aspects, the present invention provides methods of treating an aorticorenal ganglion (ARG). In certain aspects, the present invention provides methods of ablating an ARG.

### CROSS-REFERENCE TO RELATED APPLICATIONS

This application claims priority to U.S. Provisional Patent Application No. 63/146,962 filed Feb. 8, 2021, the contents of which are incorporated by reference herein in its entirety.

### STATEMENT REGARDING FEDERALLY SPONSORED RESEARCH OR DEVELOPMENT

This invention was made with government support under Grant No. DP2HL14204501 awarded by the National Institutes of Health/National Heart, Lung, and Blood Institute. The Government has certain rights in the invention.

### BACKGROUND OF THE INVENTION

The utility of renal sympathetic nerve denervation (RND) has been reported in several studies and established as one of the treatments for resistant hypertension and heart failure (Townsend R R et al., The Lancet. 2017 Nov. 11; 390 (10108): 2160-70; Kandzari D E et al., The Lancet. 2018 Jun. 9; 391 (10137): 2346-55; Sharp T E et al., Journal of the American College of Cardiology. 2018 Nov. 27; 72 (21): 2609-21. Conventional renal denervation is performed by ablating the renal nerve fibers that run horizontal to the renal artery (RA), which consists of both efferent and afferent fibers. Renal efferent fibers evoke the activity of the renin-angiotensin (R-A) system and renal sodium retention, as well as renal vasoconstriction, whereas renal afferent fibers integrate to the hypothalamic paraventricular nucleus (PVN), where they impact the central sympathetic outflow, and adjust the sympathoexcitation (Xu B et al., American Journal of Physiology-Heart and Circulatory Physiology. 2015 May 1; 308 (9): H1103-11).

The strong relationship of sympathetic nervous system activation and the occurrence of ventricular arrhythmia is well known. In addition, the utility of renal denervation in ventricular arrhythmia has been shown in several studies (Zhang W H et al., Journal of the American Heart Association. 2018 Oct. 16; 7 (20): e009938; Bradfield J S et al., Heart Rhythm. 2020 Feb. 1; 17 (2): 220-7; Chang S N et al., JACC: Basic to Translational Science. 2017 Apr. 1; 2 (2): 184-93). Regarding the investigations of optimal renal artery ablation, recent studies suggest that targeting the bifurcation, or distal area of the RA, is preferred (Hopper I et al., Journal of Cardiac Failure. 2017 Sep. 1; 23 (9): 702-7; Mahfoud F et al., Journal of the American College of Cardiology. 2015 Oct. 20; 66 (16): 1766-75; Pekarskiy S E et al., Journal of hypertension. 2017 Feb. 1; 35 (2): 369-75), and stimulating the RA could also improve the selection of the ablation sites (Chinushi M et al., Hypertension. 2013 Feb; 61 (2): 450-6). Albeit, due to undesirable outcomes between RND amongst the general population (Bhatt D L et al., N Engl J Med. 2014 Apr. 10; 370:1393-401) and in particular, heart failure patients (Hopper I et al., Journal of Cardiac Failure. 2017 Sep. 1; 23 (9): 702-7); the issues surrounding incomplete renal nerve ablation remain.

There is a need in the art for improved methods of reducing systemic adrenergic activation through renal denervation. The present invention meets this need.

### SUMMARY OF THE INVENTION

In one aspect, the present invention relates to a method of renal denervation, comprising the step of administering ablation therapy to an aorticorenal ganglion (ARG).

In one embodiment, the ablation therapy is performed endovascularly through the inferior vena cava and renal vein. In one embodiment, the ARG is selected from a left ARG, a right ARG, or both. In one embodiment, ablation therapy is applied to the left ARG at a location positioned adjacent to a superior mesenteric ganglion and on a posterior side of a left renal vein. In one embodiment, ablation therapy is applied to the right ARG at a location positioned between an inferior vena cava and descending aorta, superior to a right renal artery, on a posterior side of a right renal vein.

In one embodiment, the ablation therapy is selected from the group consisting of: cryoablation, radiofrequency ablation, chemical ablation, and laser ablation. In one embodiment, the ablation therapy is radiofrequency ablation at 20 W using an irrigated catheter with a flow rate of 8 mL/second, for at least 30 seconds.

In one embodiment, the method further comprises a step of recording a baseline hemodynamic response before the step of administering ablation therapy. In one embodiment, the step of administering ablation therapy generates an increase in hemodynamic response. In one embodiment, the method further comprises a step of ceasing ablation therapy once the hemodynamic response returns to baseline. In one embodiment, the hemodynamic response is heart rate, blood pressure, or both.

In one embodiment, the method is effective in treating systemic adrenergic activation. In one embodiment, the method is effective in treating a cardiovascular disease or disorder. In one embodiment, the cardiovascular disease or disorder is selected from the group consisting of: atrial fibrillation (AF), ventricular arrhythmia, ventricular tachycardia, systolic heart failure (reduced ejection heart failure), diastolic heart failure (preserved ejection heart failure), myocardial infarction, hypertrophy, and hypertension.

In one embodiment, the ablation therapy suppresses sympathetic nerve activation. In one embodiment, the ablation therapy suppresses effect of renal artery stimulation. In one embodiment, the method is configure to modulate ventricular arrhythmia.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts a flowchart of an exemplary method of renal neuromodulation.

FIG. 2A through FIG. 2I depicts the results of percutaneous identification of the aorticorenal ganglion (ARG) (gross and fluoroscopic anatomy), H&E of ARG. (FIG. 2A) Gross anatomy of the ARG. The Right ARG (R-ARG) is located at the border of inferior vena cava (IVC) and descending aorta, with height between the right renal artery (R-RA) and right renal vein (R-RV) ostium. The left ARG (L-ARG) is located at the posterior side on top of the left renal vein (L-RV). (FIG. 2B) The image after the IVC and the renal vein were excluded. The R-ARG is located at the right side of the descending aorta and near the ostium of the R-RA. The blue arrows portray the right renal nerve integrating into the R-ARG. The L-ARG is located on the left side of the descending aorta and inferior to the superior mesenteric artery (SMA). The yellow arrows portray the left renal nerve integrating to the L-ARG. (FIG. 2C) A fluoroscopy image portraying renal artery stimulation and ablation sites. The best response area was examined by stimulating the renal artery, annotated in yellow, and RA ablation was performed by ablating both proximal and distal side of this area, annotated in red. (FIG. 2D) Fluoroscopy image of the ARG. The R-ARG can be stimulated and ablated from the posterior side of the IVC, at the region between the R-RA ostium and R-RV ostium. The L-ARG can be stimulated and ablated at the top of the L-RV ostium. The yellow mark portrays the optimal response area for ARGs and their ablation area. (FIG. 2E) H&E staining image of ARG. The ARG consists of a large number of neurons, as indicated by the arrows, as well as the nerve fibers running in-between them. (FIG. 2F) Gross anatomy around ARGs in the pig, after L-ARG ablation (top panel) and after R-ARG (bottom panels). (FIG. 2G and FIG. 2H) Anatomic maps with CARTO focusing on ARG ablation (FIG. 2G) and RA ablation (FIG. 2H) sites. (FIG. 2I) Gross anatomy overlaid by anatomic CARTO map. Ao; Descending aorta; IVC: inferior vena cava; L: left; R: right; RV: renal vein; SMA: superior mesenteric artery.

FIG. 3A through FIG. 3C depict images demonstrating that the ARG consists almost entirely of adrenergic neurons, with evidence of cholinergic input, which pass through afferent fibers. (FIG. 3A) A representative image portraying the adrenergic neurons present in the ARG. All the ARG neurons express both anti-tyrosine hydroxylase (TH) and anti-neuropeptide Y (NPY); a marker of adrenergic neurons. (FIG. 3B) Cholinergic inputs were confirmed by vesicular acetylcholine transporter (VAChT)-expressing nerve fibers, representing cholinergic nerve fibers. Cholinergic nerve fibers pass through the ARG adrenergic neurons (Right figure in the upper row), and synaptophysin (Syn) expression was also confirmed collocating with VAChT nerve fibers (Left figure in the bottom row), indicating the VAChT-expressing nerve fiber as cholinergic transporters. (FIG. 3C) Sensory afferent nerve fibers are represented by calcitonin gene-related peptide (CGRP) expression, which also pass through the adrenergic neurons.

FIG. 4A and FIG. 4B depict the results of studies demonstrating the influence of anesthesia on ARG and RA stimulation. No hemodynamic change in renal artery stimulation (FIG. 4A) and ARG stimulation (FIG. 4B) in sedating with isoflurane.

FIG. 5A through FIG. 5G depict the results of studies demonstrating the ARG yields larger hemodynamic responses than renal artery stimulation via larger catecholamine release. Hemodynamic changes during the renal artery stimulation (RAs;

FIG. 5A) and ARG stimulation (ARGs; FIG. 5B). ARG stimulation showed a significantly greater increase in comparison to RNS in Systolic BP (SBP), Diastolic BP (DBP) and HR (FIG. 5C). (FIG. 5D) ARI changes related to both renal artery stimulation (RAs) and ARG stimulation (ARGs). The actual ARI change (left) and ARI-corrected by Bazett's formula (right). (FIG. 5E) Plasma catecholamine levels at baseline and following RAs and ARGs. ARGs showed a significantly greater increase in comparison to RAs, in both norepinephrine and epinephrine levels. (FIG. 5F, FIG. 5G) Changes of firing frequency of SG due to RAs and ARGs stimulation. ARI; activation recovery interval, BP; blood pressure, EKG; electrocardiogram, HR; heart rate, SG; stellate ganglion. *p<0.05, **p<0.01.

FIG. 6 is a table listing the changes in systolic blood pressure (SBP), diastolic blood pressure (DBP), and heart rate (HR) before and after stimulation of the left and right renal artery and left and right aorticorenal ganglion.

FIG. 7A through FIG. 7G depict the results of studies demonstrating ARG ablation eliminates hemodynamic responses to both renal artery stimulation and ARG stimulation. Hemodynamic changes during the ablation. No significant change in hemodynamic data was observed during renal artery ablation (FIG. 7A). In the post-renal artery ablation case, no response was observed in renal artery stimulation, while ARG stimulation still occurred (FIG. 7B, FIG. 7G). Whereas ablating the ARG locating area causes a marked increase in BP (FIG. 7D). After ARG ablation, both RNS and ARG stimulation showed no response in hemodynamic data (FIG. 7E, FIG. 7F). (FIG. 7G) Summary of % change of systolic BP (% SBP) with renal artery stimulation and ARG stimulation. This demonstrates that isolation of renal afferent pathways can be achieved by the ARG ablation. Abbreviations are the same in FIG. 5A through FIG. 5G.

FIG. 8A through FIG. 8C depict the results of studies demonstrating that ARG ablation does not impact adrenal glands or kidneys acutely. No significant differences in renal tissue histology between control (FIG. 8A) and ablation groups (FIG. 8B) were observed. Each panel displays trichrome stained renal tissue at low power (left: 40×, scale bar 400 um) and high power (right: 200×, scale bar 100 um) magnification. Solid arrows=glomeruli. Dashed arrows=interlobular arteries. (FIG. 8C) No significant differences in adrenal tissue histology between control and ablation groups were observed. Each panel displays hematoxylin and Eosin stained adrenal cortical tissue at low power (left: 40×) and high power (right: 200×) magnification.

FIG. 9A through FIG. 9F depicts aorticorenal ganglion (ARG) and renal artery (RA) ablation eliminate neurons and nerves. (FIG. 9A) Gross and hematoxylin and eosin (H&E) stained images of nonablated ARG. Dotted rectangle shows an expanded view. Arrows indicate neurons. (FIG. 9B) Representative whole immunohistochemistry (IHC) images of nonablated ARG with tyrosine hydroxylase (TH) and neuropeptide Y (NPY). Dotted rectangles (top panels) are shown in expanded view (bottom panels). (FIG. 9C) Gross and H&E stained mages of ablated ARG. Dotted rectangle shown an expanded view. There were no neurons in the ablated ARG. (FIG. 9D) Representative whole IHC images of ablated ARG with TH and NPY. Dotted rectangle shows an expanded view. (FIG. 9E and FIG. 9F) H&E stained images of nonablated RA (FIG. 9E) and abated RA (FIG. 9F). Dotted rectangle show an expanded view. Arrows indicate nerve fibers.

FIG. 10A through FIG. 10F depict the results of studies demonstrating ARG ablation prevents ischemia-induced VF by suppressing sympathoexcitation. (FIG. 10A) LAD occlusion (LAD occ) was performed at the proximal side of the bifurcation at the 2nd diagonal branch. (FIG. 10B) Hemodynamic changes of LAD occlusion. SBP decrease due to LAD occlusion had no difference between the 3 groups, while HR did. The ARG ablation group showed a significantly weaker response compared to the control and renal artery ablation group. (FIG. 10C) The control and RA ablation cases had frequent occurrences of VF and died (upper image), but the ARG ablation cases show recovery from ventricular arrhythmia and survival (lower image). (FIG. 10D, FIG. 10E) The occurrence of ventricular arrhythmia in the 3 groups. The ARG ablation group had a significantly longer interval for both PVC and the first VT or VF episode (FIG. 10D) and showed a significantly lower occurrence of VF (FIG. 10E). (FIG. 10F) Electrophysiological parameters change during the LAD occlusion. The ARG group showed significantly smaller changes in both corrected-RT and corrected-ARI, compared to the control and RA ablation group. LAD; left anterior descending artery, PVC; premature ventricular construction, VF; ventricular fibrillation, VT; ventricular tachycardia, *p<0.05. **p<0.01.

FIG. 11A through FIG. 11I depict the anatomy and physiology of the human ARG. (FIG. 11A) Gross anatomy around ARGs in the human. (FIG. 11B, FIG. 11C) The L-ARG is located posterior to the L-RV and next to the SMA. (FIG. 11D, FIG. 11E) The R-ARG is located between the IVC and descending aorta, and superior to R-RA. (FIG. 11F) The image just after the intestines were removed. (FIG. 11G, FIG. 11H) H&E staining image of ARGs. Both right and left ARGs consist largely of neurons, as indicated by the arrows. (FIG. 11I) A representative immunofluorescence of human ARG using TH and NPY; a marker of adrenergic neurons. IMA; inferior mesenteric artery, IMV; inferior mesenteric vein, other abbreviations are the same as shown in FIG. 2A through FIG. 2I.

FIG. 12 is a schematic showing the sympathetic nerve system and ARG. The ARG has the post-ganglionic sympathetic neurons that innervate the kidney and is also directly linked to the renal plexus which consist of renal nerve.

FIG. 13 depicts a right renal artery (left) and the effect of an isoflurane anesthetic and a cholarolose-based anesthetic on systolic blood pressure to determine an anesthetic that allows for robust hemodynamic responses in stimulation the renal artery.

FIG. 14A and FIG. 14B show the results of experiments determining sites for reliable renal artery stimulation. FIG. 14A shows examples of site demarcation of the right and left renal artery. FIG. 14B shows the effects of different stimulation approaches.

FIG. 15 shows the locations of renal nerve ablation (top) and the differences in heart rate (HR) and left ventricular pressure (LVP) during renal nerve stimulation before and after ablation.

FIG. 16 shows the results of immunohistochemistry performed at a renal artery bifurcation, showing several large nerve bundles and fibers that are predominantly adrenergic.

FIG. 17 shows the results of stimulating the ARG in comparison to the renal artery.

FIG. 18 shows the results of validating endovascular approaches to target the ARG. Fluoroscopy-identified endovascular sites (top) were matched with gross anatomy (bottom).

FIG. 19 shows the results of immunohistochemistry performed on the ARG to confirm that the ARG contains post-ganglionic adrenergic neurons.

FIG. 20 shows the results of enzyme-linked immunosorbent assays for noradrenaline and adrenaline for the following conditions: Baseline (Pre); renal artery (RA), Aorticorenal ganglion (ARG), coronary artery occlusion (CA).

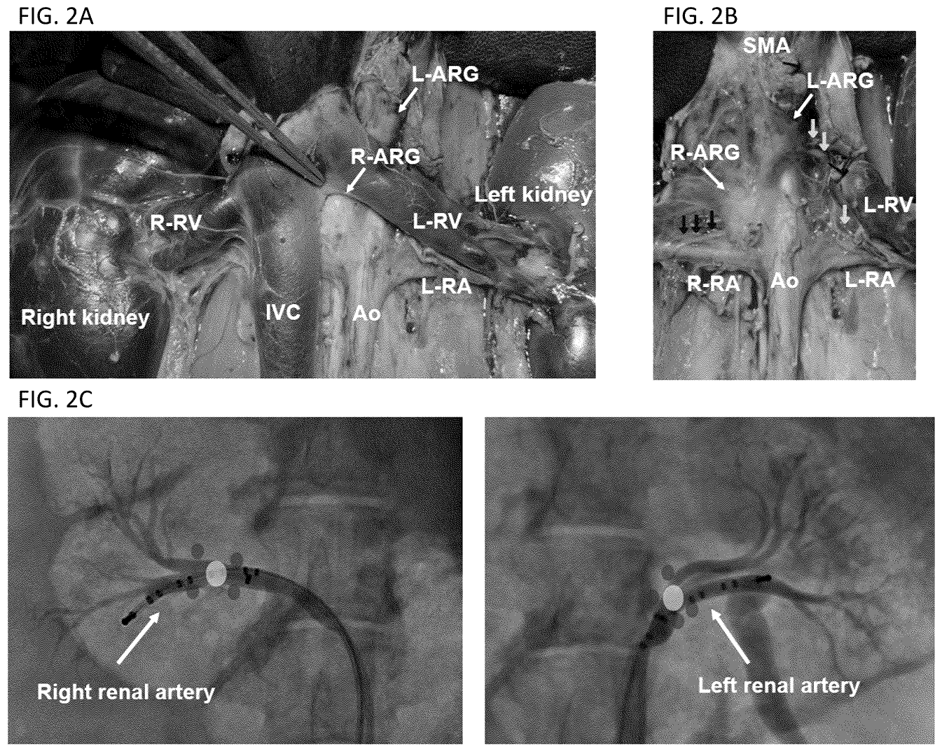

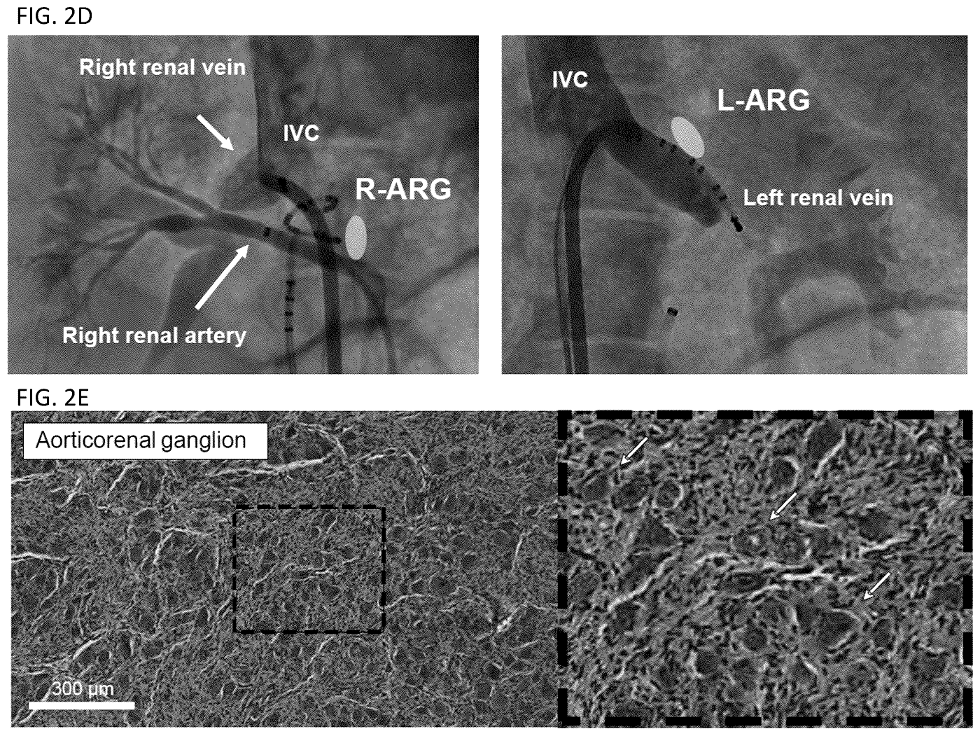

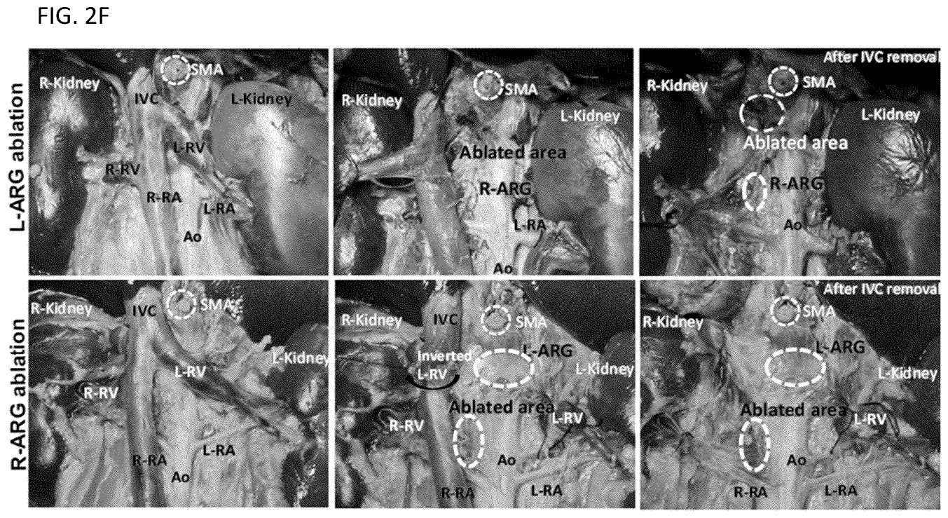

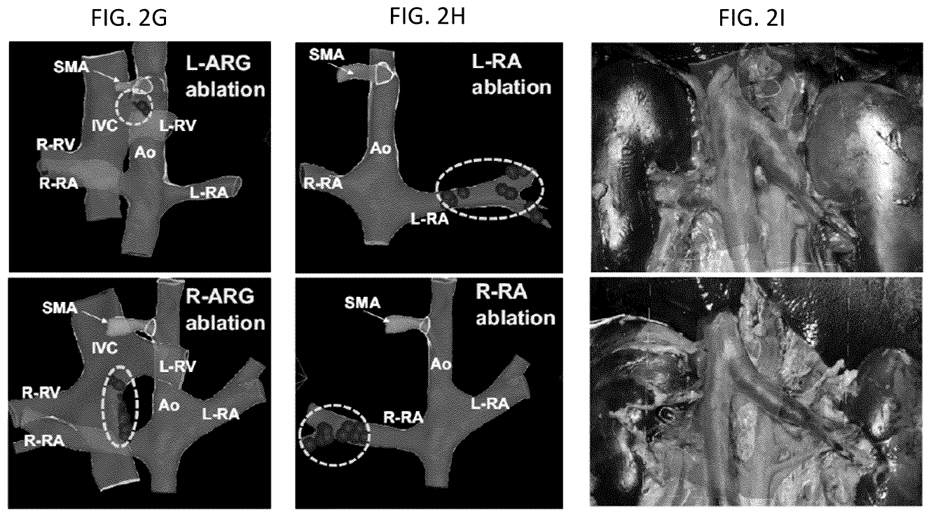

FIG. 2I shows the results of measuring corrected activation recovery intervals (cARI) in response to ARG and renal artery stimulation.

FIG. 22 shows the results of ablation and subsequent stimulation of the ARG on heart rate and left ventricular pressure.

### DETAILED DESCRIPTION

The present invention provides methods for renal denervation for the effective treatment of systemic adrenergic activation and related diseases and disorders, including but not limited to cardiovascular diseases. In certain aspects, the present invention provides methods of treating an aorticorenal ganglion (ARG). In certain aspects, the present invention provides methods of ablating an ARG.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

The term “biomolecule” or “bioorganic molecule” refers to an organic molecule typically made by living organisms. This includes, for example, molecules comprising nucleotides, amino acids, sugars, fatty acids, steroids, nucleic acids, polypeptides, peptides, peptide fragments, carbohydrates, lipids, and combinations of these (e.g., glycoproteins, ribonucleoproteins, lipoproteins, or the like).

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

As used herein, the term “composition” or “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease, or disorder, the frequency with which such a symptom is experienced by a patient, or both, are reduced.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

“Extracellular matrix” or “matrix” refers to one or more substances that provide substantially the same conditions for supporting cell growth as provided by an extracellular matrix synthesized by feeder cells. The matrix may be provided on a substrate. Alternatively, the component(s) comprising the matrix may be provided in solution. Components of an extracellular matrix can include laminin, collagen and fibronectin.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, “scaffold” refers to a structure comprising a biocompatible material that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

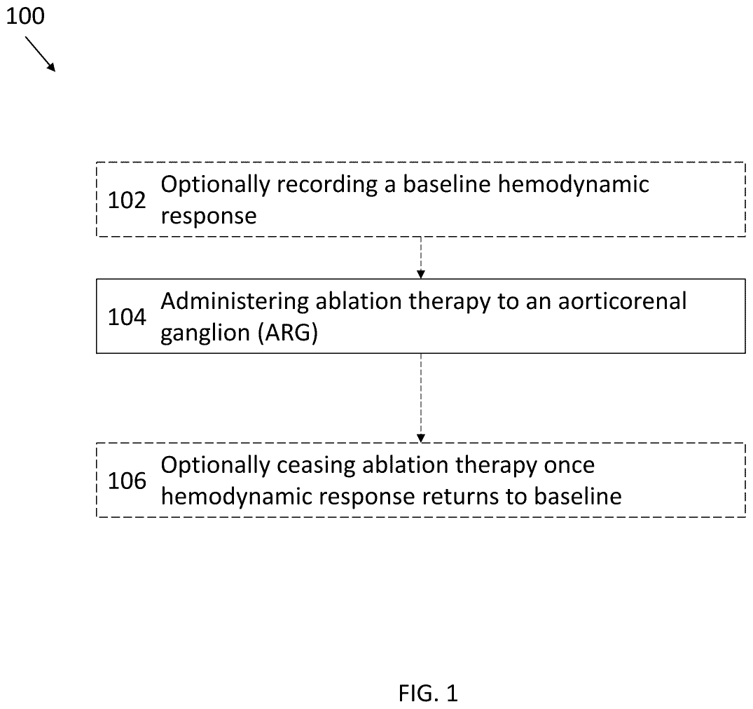

Referring now to FIG. 1, an exemplary method 100 is depicted. Method 100 comprises a step 104 of administering ablation therapy to an aorticorenal ganglion (ARG). In some embodiments, the ablation therapy is applied to a left ARG, a right ARG, or both. The ablation therapy can be selected from cryoablation, radiofrequency ablation, chemical ablation, and laser ablation. In some embodiments, radiofrequency ablation is applied. Radiofrequency ablation can be applied at about 1 W to about 50 W, or about 20 W using an irrigation catheter having a flow rate of between about 1 mL/second to about 50 mL/second, or about 8 mL/second. Ablation therapy can be applied for any suitable period of time, such as between about 30 seconds to about 400 seconds.

Ablation therapy can be applied through any desired approach. In some embodiments, ablation therapy is applied endovascularly, such as through the inferior vena cava and renal vein. For example, ablation therapy can be applied to the left ARG at a location positioned adjacent to a superior mesenteric ganglion and on a posterior side of a left renal vein. Ablation therapy can be applied to the right ARG at a location positioned between an inferior vena cava and descending aorta, superior to a right renal artery, on a posterior side of a right renal vein.

In some embodiments, method 100 comprises an optional step 102 preceding step 104, wherein in step 102 a baseline hemodynamic response is recorded. The hemodynamic response can be any suitable hemodynamic response, such as heart rate, diastolic blood pressure, and systolic blood pressure. Baseline hemodynamic conditions are measured at under basal or resting conditions. ARG stimulation, including ablation therapy, may lead to spikes in hemodynamic response, such as increased heart rate and blood pressure. Complete ablation of the ARG can thereby be indicated by a cessation of raised levels of hemodynamic response, such as a return to baseline. Accordingly, method 100 can comprise an optional step 106 after step 104, wherein in step 106 ablation therapy is ceased once hemodynamic response returns to baseline.

The methods of the present invention can be used to treat or prevent one or more cardiovascular diseases or disorders, including but not limited to atrial fibrillation (AF), ventricular arrhythmia, ventricular tachycardia, ischemia, systolic heart failure (reduced ejection heart failure), diastolic heart failure (preserved ejection heart failure), myocardial infarction, hypertrophy, and hypertension in a subject in need thereof. In certain embodiments, the methods can reduce excessive systemic sympathetic input or excessive sympathetic input to the heart.

In one embodiment, the present invention comprises a system for accomplishing ablation therapy. In one embodiment, the ablation therapy may be performed using any tool known to one skilled in the art including but not limited to an ablation catheter. In one embodiment, the ablation tool may be an irrigated ablation catheter.

In one embodiment, the present invention comprises a system for accomplishing recording the baseline hemodynamic response. In one embodiment, the system may comprise any device known to one skilled in the art that is able to measure at least one hemodynamic response.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art may, using the preceding description and the following illustrative examples, make and utilize the compounds of the present invention and practice the claimed methods. The following working examples therefore specifically point out exemplary embodiments of the present invention, and are not to be construed as limiting in any way the remainder of the disclosure.

### Example 1: The Aorticorenal Ganglion as a Novel Target for Renal Neuromodulation

The aorticorenal ganglion (ARG) is located superior to the renal arteries, and just lateral to the descending aorta, and produces fibers that innervate the kidneys. Thus, the ARG presents itself as a novel upstream target for renal sympathetic nerve fibers. The ARG plays an important role in innervating both tubular and vascular components of the kidney (Norvell J E, Journal of Comparative Neurology. 1968 May; 133 (1): 101-11). A recent study found the endpoint of RND through the ablation of ARG pace capture sites (Qian P C et al., JACC: Cardiovascular Interventions. 2019 Jun. 24; 12 (12): 1109-20), however, the ARG has not been specifically targeted for renal denervation. The present invention demonstrates that ARG ablation results in more complete renal denervation and greater suppression of sympathetic activation, especially when compared to renal artery ablation alone.

The materials and methods are now described.

### Animal Studies

A total of 26 Yorkshire pigs (S&S farms) of either sex (60-70 kg) were studied acutely. Sedation was performed by administering Telazol (5-8 mg/kg) intramuscularly. The subjects were then intubated and maintained on general anesthesia with isoflurane (1-3%) during the surgical preparation. Temperature was maintained at 36-38° C., a 12-lead ECG was recorded (GE CardioLab), saline was provided at a continuous rate of 6-8 ml·kg⁻¹·h⁻¹, and arterial access was gained to measure blood and left ventricular pressure readings (Millar, Houston, TX). A midline sternotomy was subsequently performed to expose the heart and left stellate ganglia (L-SG). After the surgical preparation was completed a transition to alpha-chloralose (6.25 mg/125 mL, 1 ml/kg bolus for 30-60 minutes, with a sustained dose of 25-35 ml/kg/h) took place and was maintained for the remainder of the protocol. Furthermore, an additional 30 minutes was spent after the alpha-chloralose bolus was completed to ensure the pig was stabilized before beginning the three experimental protocols.

### RA and ARG Stimulation

The first protocol investigated the stimulation of the RA and ARG in porcine subjects (n=10) to find the optimal region of stimulation that would result in a consistent and significant hemodynamic response. All stimulation parameters were set at 20 Hz and 15 mA (5 msec) for a total of 60 seconds, with the best response recorded for each side. Renal artery stimulation was performed by inserting a 10-electrode duodecapolar catheter at the proximal, bifurcation, and distal areas of the renal artery for stimulation (Abbott, MN, USA). The ARGs are located upstream of both renal nerves, running parallel to each renal artery and diverging from the artery cranially to converge with the larger ARG's. The Right-ARG (R-ARG) is located in the area between the inferior vena cava (IVC) and descending aorta, and between the R-RA and right renal vein (R-RV) ostium (FIG. 2A, FIG. 2B). The Left-ARG (L-ARG) is observed under the superior mesenteric artery (SMA), and posterior to the top segment of the left renal vein (L-RV) (FIG. 2A, FIG. 2B). R-ARG stimulation was performed by placing 20 electrode circular mapping catheter (Inquiry™ Electrophysiology Catheters 7F, Abbott) in the IVC. L-ARG stimulation was performed by inserting a duodecapolar catheter in the left renal vein (L-RV) and stimulating the posterior region of the L-RV proximal site (FIG. 2D). To aid interventional approaches to the ARGs, a 3-dimensional electroanatomic mapping system (CARTO system, Biosense Webster, Diamond Bar, CA) was used. The gross anatomic, electroanatomic mapping, and fused images are shown in FIG. 2F through FIG. 2I.

### RA and ARG Ablation

The second protocol involved randomly assigning the remaining 16 subjects into 3 groups: Control (n=5), RA ablation (n=5), and ARG ablation (n=6) to investigate the impact of renal denervation during acute ischemic stress. Renal artery ablation was performed by ablating the proximal and distal sites of the sites showing the best hemodynamic responses to stimulation (FIG. 2C). Each site was ablated with an irrigated ablation catheter (SMARTTOUCH, Biosense Webster, Diamond Bar, CA, USA), with each ablation being set to 25 W, with a flow of 20 ml/min, for 30-60 seconds. The best responses were observed at the distal bifurcation of the renal artery. In those instances, ablation was performed at the bifurcation, both branches after the bifurcation, and the top and bottom portions of the artery before the bifurcation, for a total of 5 ablation sites. All ARG ablation cases were accomplished by approaching from the venous system only, through the IVC, and confirmed by visualizing a notable BP increase (greater than 30% increase during electrical stimulation) from the procedure. ARG ablation ceased when responses in the BP were extinguished during ablation events. Stimulation was conducted after ablation to confirm the success of RA or ARG ablation. If a hemodynamic response to stimulation remained, ablation was repeated until the BP change was abolished.

Comparing ARG Vs. RA Ablation During Acute Ischemia

The third and final component to the protocol involved the creation of an ischemia-induced ventricular arrhythmia heart model by acutely occluding the Left Anterior Descending (LAD) artery to compare the effects of renal denervation after ablation. Acute Ischemia was created by ligating an isolated area of the (LAD) just proximal to the 2nd diagonal branch. The LAD occlusion (LAD occ) was continued for 15 minutes, followed by a release of the ligature and an additional 15 minutes for reperfusion. To compare the effect of renal denervation on ventricular arrhythmogenicity, time to frequent premature ventricular contractions (PVC) (defined as PVC burden equal to 4:1 or greater), ventricular tachycardia (VT), and ventricular fibrillation (VF) was examined by using the Kaplan-Meyer method. To investigate the mechanisms by which renal denervation reduces ventricular arrhythmogenesis, repolarization time (RT) and activation recovery interval (ARI) were examined as a surrogate for action potential duration adjacent to the ischemic region. Catecholamine spill-over was also examined, and cardiac sympathetic signaling was sampled by measuring stellate ganglia (SG) neural activity in vivo in sham, RA, and ARG ablation groups.

Stellate ganglion activity and cardiac EP measures

To record sympathetic outflow during stimulations and LAD occlusion, a 6-channel linear microelectrode array (MICROPROBES, Gaithersburg, MD) was carefully placed into the craniomedial aspect of the left SG at an angle horizontal to the ganglion (Beaumont E et al., The Journal of physiology. 2013 Sep; 591 (18): 4515-33; Yoshie K et al., American Journal of Physiology-Heart and Circulatory Physiology. 2018 May 1; 314 (5): H954-66; Yoshie K et al., JCI insight. 2020 Feb. 13; 5 (3)). Signals were recorded using a microelectrode amplifier, with data preamplified via a headstage (model 3600, A-M Systems, Carlsborg, WA). Signals were filtered at 300 Hz to 3 kHz, amplified with a gain of 500-2000, and were recorded continuously alongside electrocardiographic (ECG) and hemodynamic data using the Spike2 application (Cambridge Electronic Design) for processing of firing frequency and further electrophysiological data analysis. Cardiac electrical mapping was performed by placing a 64-electrode array (8×8, 2 mm interelectrode spacing) on the LV wall epicardium. The electrodes were placed lateral to the 2nd diagonal LAD branch to avoid any effects ischemic change may have on data acquisition during the ligation. The recorded electrical mapping data were analyzed to calculate the myocardial RT and ARI by using a customized software, ScalDyn (Scientific Computing and Imaging Institute, University of Utah, Salt Lake City, UT).

### Cadaveric Studies

Two cadaveric specimens were dissected to localize the ARG (and RA), and specifically to examine fiber tracts connecting the ARG to other ganglia in the aortico-renal region. Ganglia were confirmed by histology and immunohistochemistry as previously described.

### H&E Staining and Immunochemistry Imaging

The tissues were rapidly excised, rinsed and washed in 0.1 M PBS, and suspended in chilled 4% paraformaldehyde for 24 hours to become fixed. They were then cleaned, embedded in paraffin, and sectioned at 5 μm thickness. Following the sectioning of tissues, Immunohistochemistry (IH) was performed on the ARG with the primary and secondary antibodies used as portrayed in Table 1. Imaging was performed with a Zeiss LSM 780 confocal microscope (Carl Zeiss, Oberkochen, Germany), stored within the Zen software (Black edition, Carl Zeiss Microimaging), and evaluated further through ImageJ. Tissue sections were also stained with hematoxylin and eosin stains as well as Masson Trichrome stains for morphologic assessment. Light microscope images were obtained on an Olympus BX53 microscope fitted with a DP27 Olympus camera via Cellsens software (Olympus life science, United States).

TABLE 1

| | Concen- | |
|---|---|---|
| Antibody | tration | Source |
| Primary antibody | | |
| Tyrosine | 1:50 | Polyclonal sheep antibody, AB1542 |
| hydroxylase | | EMD Millipore |
| Neuropeptide Y | 1:50 | Polyclonal goat antibody, NPB1-46535 |
| | | NOVUS (Centennial, CO) |
| Vesicular | 1:100 | Polyclonal rabbit antibody, 139-103 |
| acetylcholine | | Synaptic system (Germany) |
| transporter | | |
| Calcitonin | 1:50 | Monoclonal mouse antibody, ab81887 |
| gene-related | | Abcam |
| peptide | | |
| Synaptophysin 1 | 1:50 | Polyclonal chicken antibody, 101-006 |
| | | Synaptic system |
| Secondary Antibody | | |
| Alexa Fluor 488 | 1:200 | Polyclonal rabbit antibody, 711-545-152 |
| AffiniPuredonkey | | Jackson Immunoresearch Laboratories |
| anti-rabbit IgG | | (West Grove, PA) |
| Alexa Fluor 488 | 1:200 | Polyclonal sheep antibody, 713-545-003 |
| AffiniPuredonkey | | Jackson Immunoresearch Laboratories |
| anti-goat IgG | | |
| Cy3AffiniPuredonkey | 1:200 | Polyclonal mouse antibody, |
| anti-mouse IgG | | 715-165-150 |
| | | Jackson Immunoresearch Laboratories |
| Cy3AffiniPuredonkey | 1:200 | Polyclonal goat antibody, |
| anti-goat IgG | | 705-165-147 |
| | | Jackson Immunoresearch Laboratories |
| Cy3AffiniPuredonkey | 1:200 | Polyclonal rabbit antibody, |
| anti-rabbit IgG | | 711-165-152 |
| | | Jackson Immunoresearch Laboratories |
| Alexa Fluor | 1:200 | Polyclonal chicken antibody, |
| AffiniPuredonkey | | 703-605-155 |
| anti-sheep IgG | | Jackson Immunoresearch Laboratories |

### Statistical Analysis

All data are presented as mean±SEM. The Shapiro-Wilk test was used for assessing normal distribution in each group data. Group comparisons were performed with the Student's t-test for normal distribution data and the Wilcoxon signed-rank test for non-normally distributed data. When comparing the groups of 3 or greater, an ANOVA analysis was used if all the values were confirmed as normally distributed, or a Kruskal-Wallis test for non-normal distributions. For all the comparisons, p values of <0.05 were considered as statistically significant. Data were handled and analyzed using GraphPad Prism (La Jolla, CA).

The results are now described.

The ARG is a Sympathetic Ganglion with Cholinergic Inputs and Pass-Through Afferents

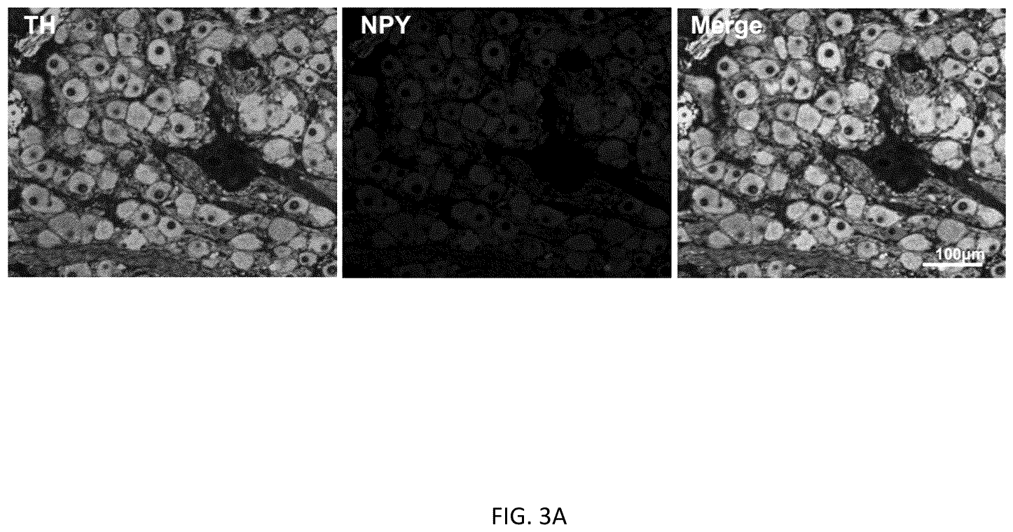

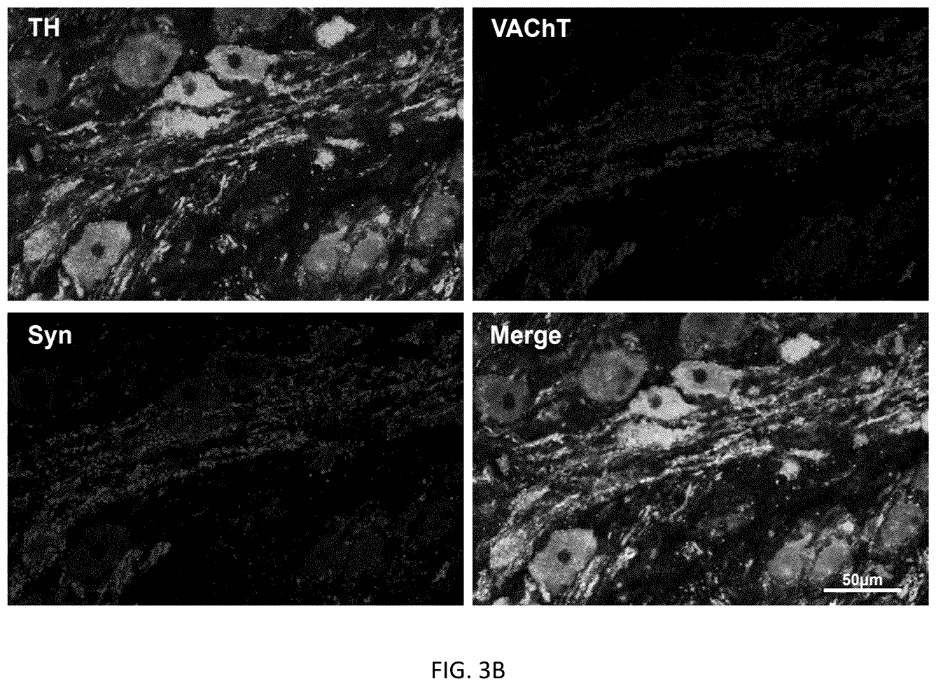

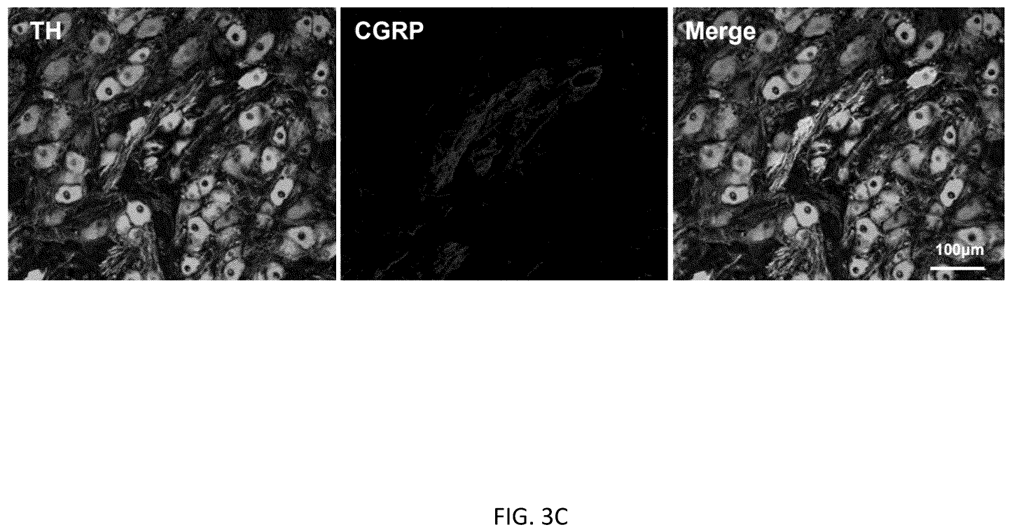

The ARG consists of adrenergic neurons, demonstrated by immunoreactivity against tyrosine hydroxylase (TH) and neuropeptide Y (NPY) (FIG. 3A) antibodies. Further, ARG demonstrated cholinergic inputs evidenced by co-localization of the vesicular acetylcholine transporter (VAChT) and synaptophysin (FIG. 3B). Pass-through afferent fibers were evaluated by the immunoreactivity to calcitonin gene-related peptide (CGRP), a sensory marker, traversing the space between adrenergic neurons (FIG. 3C).

### Anesthetic Agents Influence Hemodynamic Responses to RA and ARG Stimulations

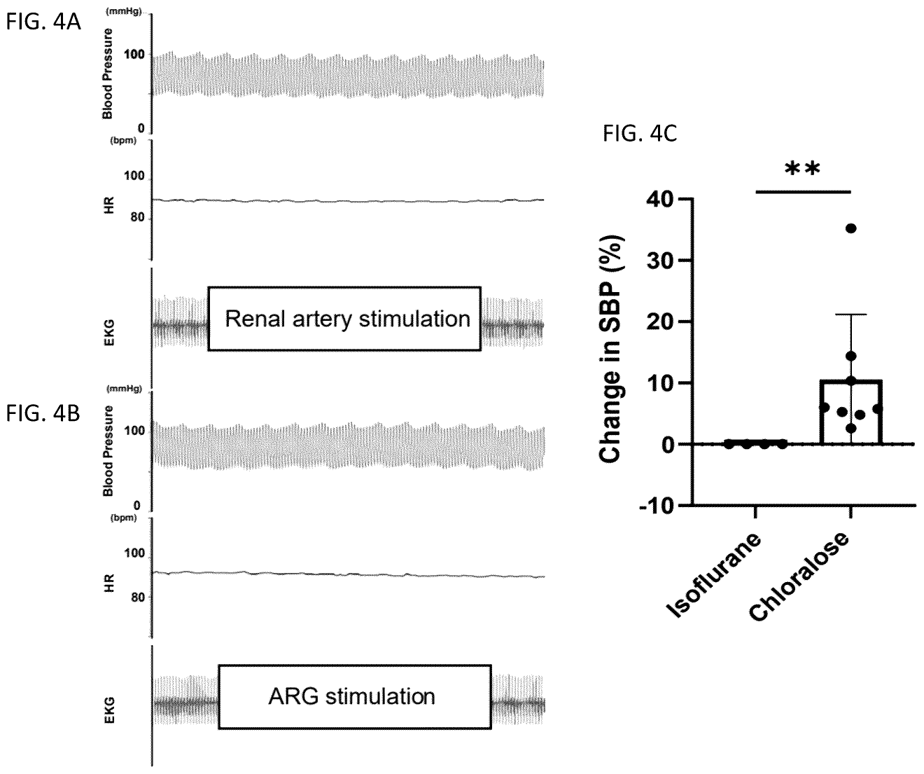

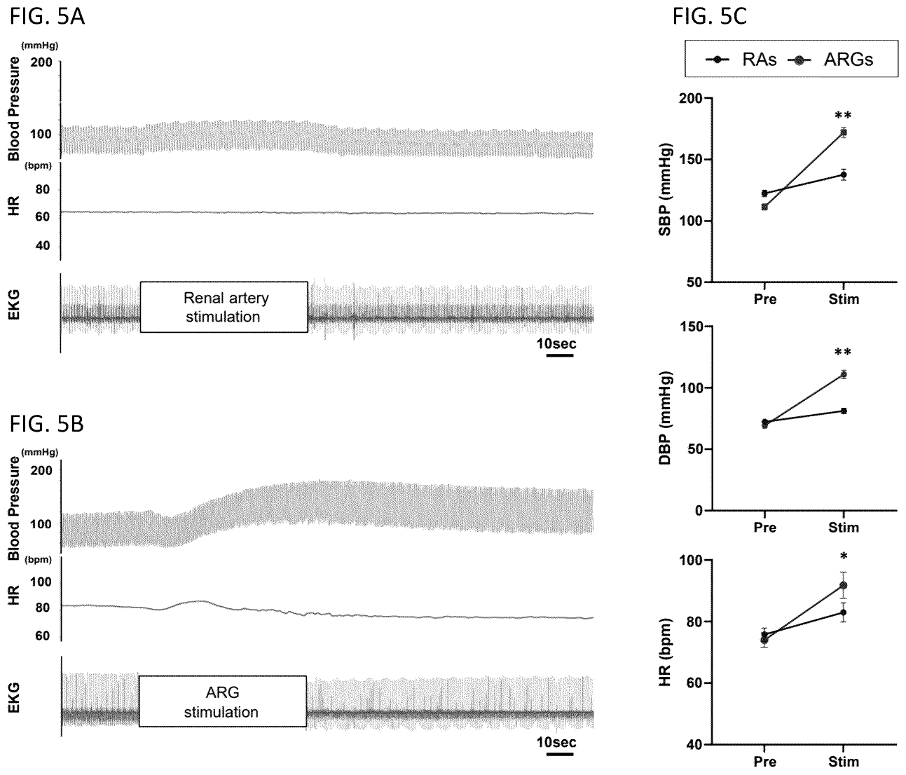

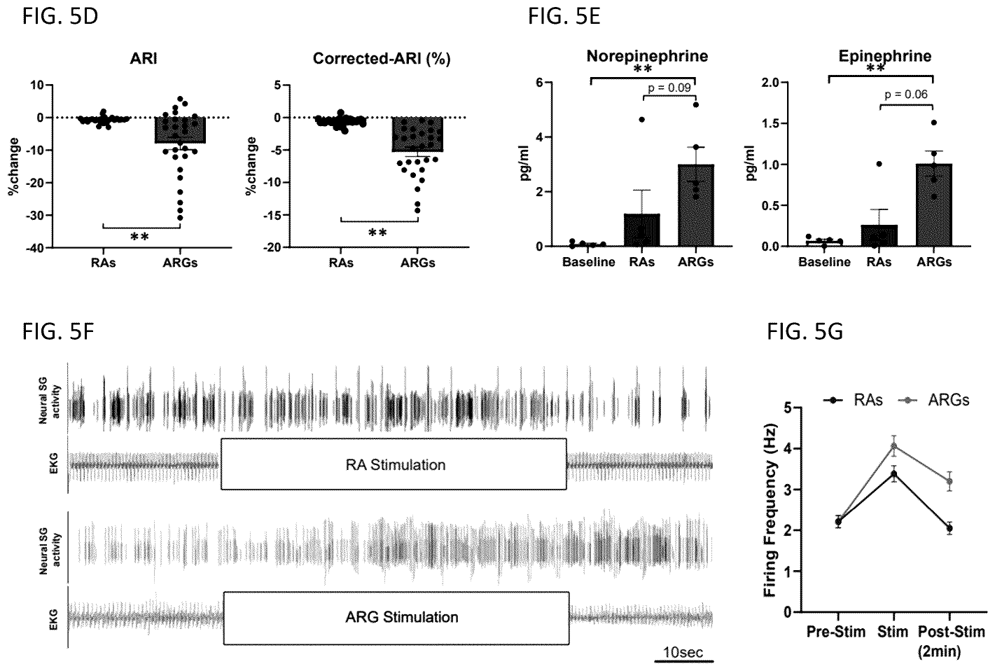

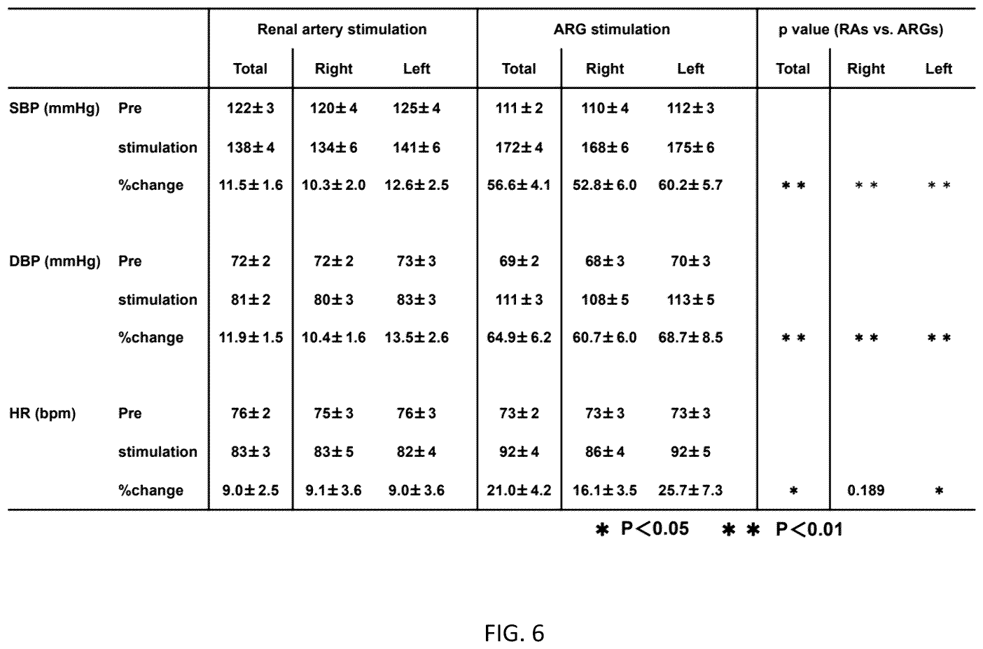

Hemodynamic responses to both RA and ARG stimulation was not present when subjects were sedated with isoflurane (FIG. 4). However, under alpha-chloralose anesthesia, ARG stimulation demonstrated a significantly greater response in systolic blood pressure (SBP), diastolic BP (DBP) and heart rate (HR) as compared to RA stimulation (FIG. 5A through FIG. 5C and FIG. 6). These responses were equivalent in both right and left ARG stimulation (FIG. 6).

Cardiac Sympathetic Activity, Catecholamine Release and Electrophysiological Parameters During ARG Vs. RA Stimulation

The effect of ARG stimulation was next assessed on the cardiac sympathetic outflow, systemic catecholamine release, and cardiac electrophysiological function compared to RA stimulation. At baseline there were no significant differences observed between the groups in terms of stellate ganglion firing frequency (ARG group vs. RA group; 2.1±0.1 vs. 2.1±0.1%, p=0.86), however in response to stimulation of the ARG or RA, the frequency of the SG neural firing increased. Specifically, peak firing increased to 3.7±0.2 Hz for ARG stimulation and 3.0±0.2 Hz for RA stimulation (p=0.06). Interestingly, SG firing frequency 2 minutes after the ARG stimulation was 3.1±0.2%, while RA stimulation was 1.9±0.1% (p<0.01), suggesting that ARG stimulation resulted in protracted sympathoexcitation compared to RA stimulation (FIG. 5F, FIG. 5G). Moreover, catecholamine release was significantly higher in the ARG stimulation group compared to the RA stimulation, which may explain the robust differences between hemodynamic responses in ARG vs. RA stimulation (FIG. 5E). Consistent with these data, ARG stimulation was associated with a greater reduction in both ARI and corrected ARI (cARI) compared to RA stimulation (% ΔARI; −7.9±1.9 vs. −0.7±0.2%, p<0.01, % ΔcARI; −5.3±0.7 vs. −0.7±0.1%, p<0.01, respectively, FIG. 5D).

ARG Ablation Eliminates the Response of Both RA and ARG Stimulation, while RA Ablation does not Impact ARG Stimulation

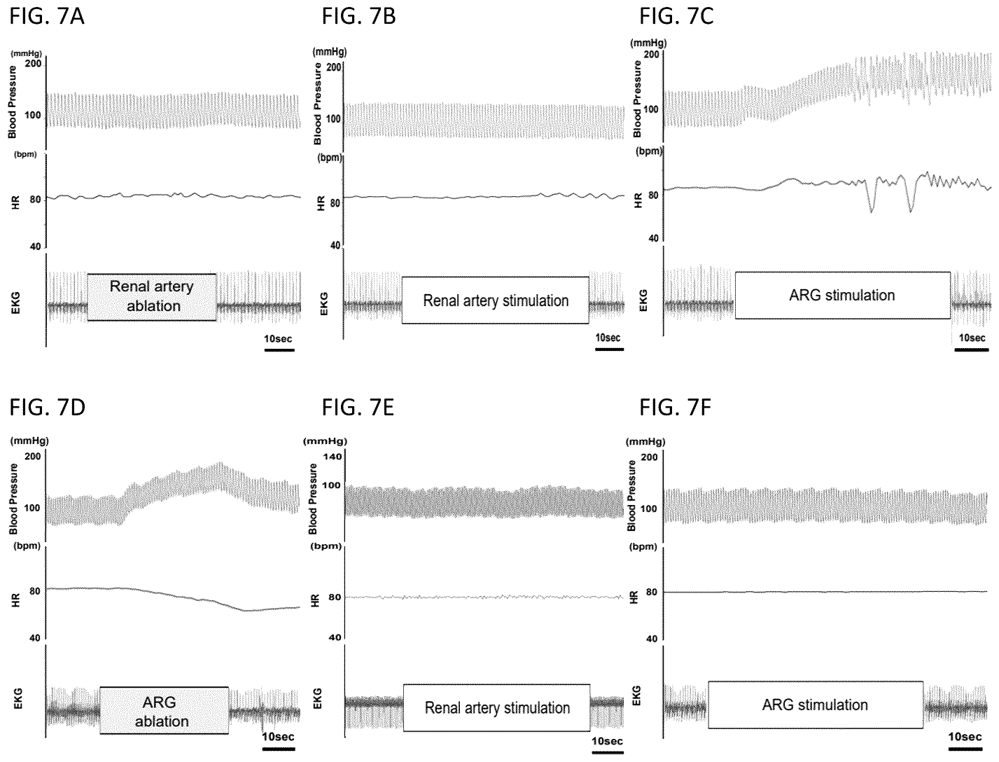

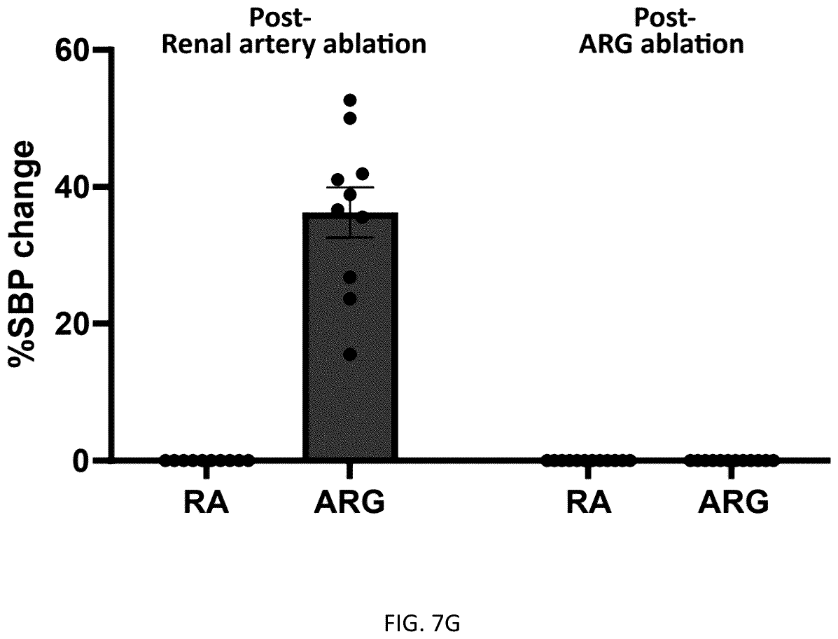

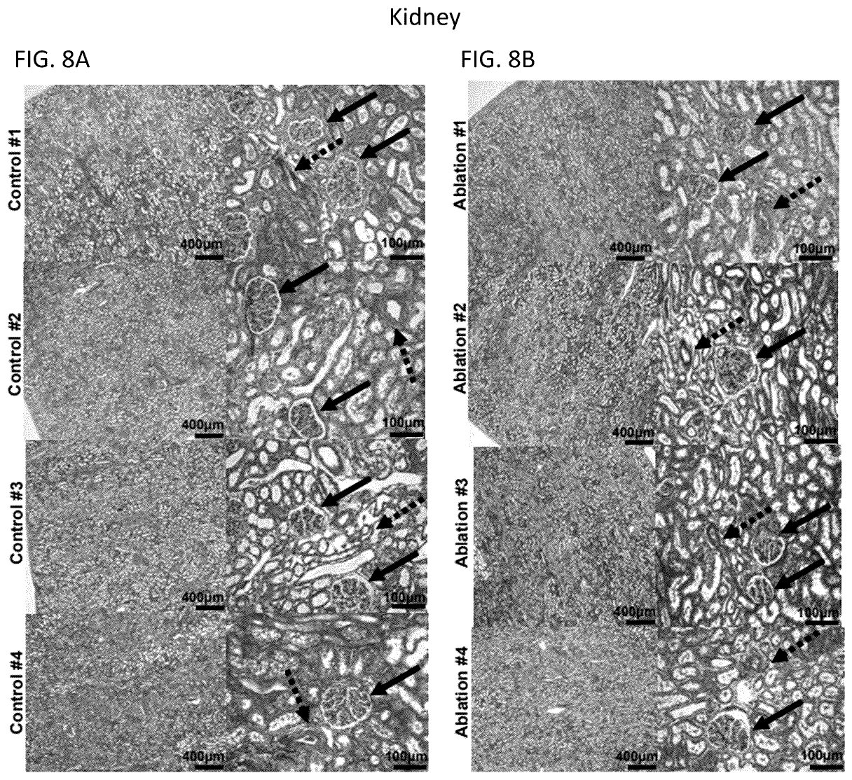

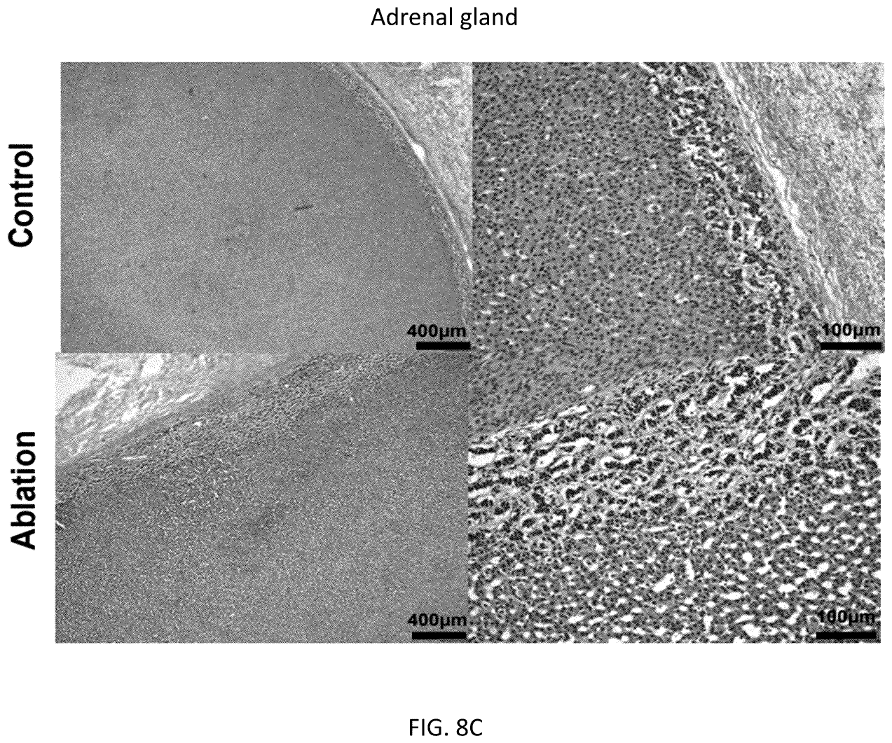

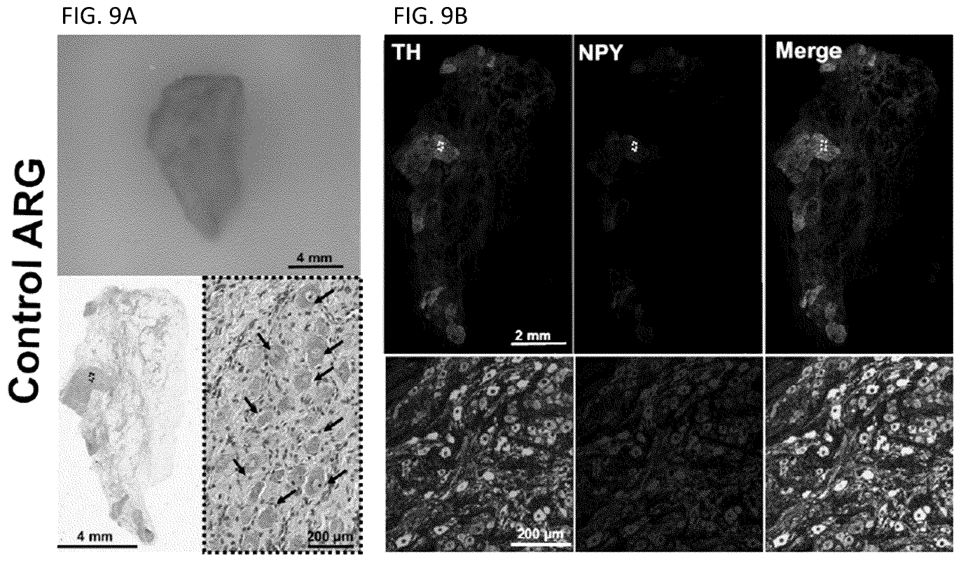

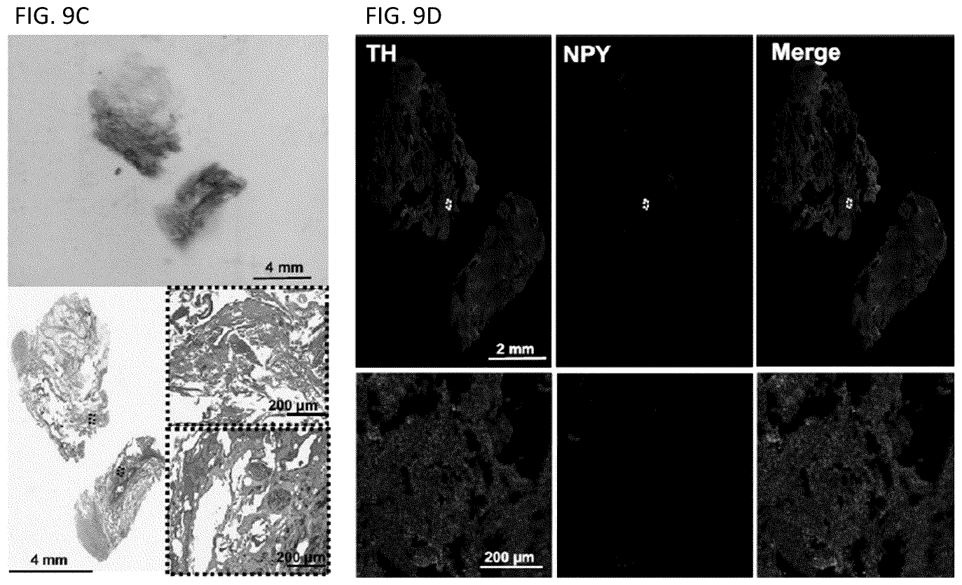

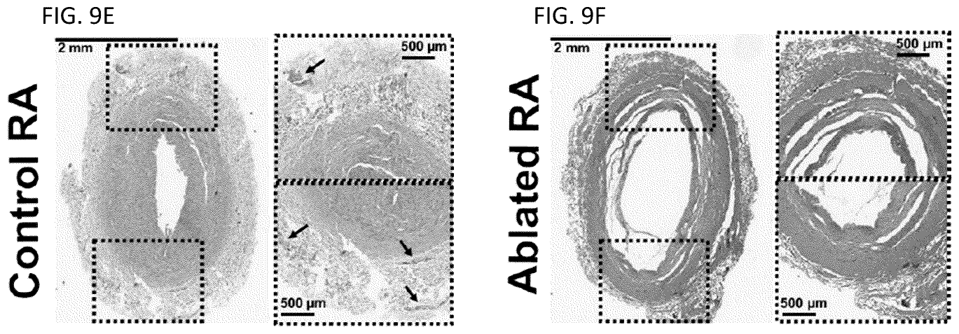

Eleven subjects were randomized to renal denervation, 5 received ARG ablation, while the remaining 6 received RA ablation. FIG. 7A and FIG. 7D show representative hemodynamic responses during RA and ARG ablation. A significant increase in BP was always seen during ARG ablation (FIG. 7D), which was not observed in the RA ablation (FIG. 7A). ARG ablation was continued until there was no response in BP (690±108 seconds). A following 60-second stimulation at the site was performed, yielding optimal responses. Absent responses at sites with initial robust responses confirm complete ablation. ARG ablation eliminated both the RA and ARG stimulation responses, whereas the RA ablation alone only eliminated RA responses (FIG. 7B, FIG. 7C, FIG. 7E, FIG. 7F). Successful ablation of the ARG and RA was confirmed via histology and IHC, and are shown in FIG. 9A through FIG. 9D for ARG ablation an in FIG. 9E and FIG. 9F for RA ablation. This suggests that ARG ablation results in more complete renal denervation than RA ablation alone. There were no procedural complications related to ablation. Moreover, the ARG ablation did not impact the kidneys or adrenal glands, as demonstrated by histologic analyses of entire kidneys and adrenal glands did not reveal any evidence of injury to these organs (FIG. 8A through FIG. 8C).

### ARG but not RA Ablation Protects Against Acute Ischemic Ventricular Arrhythmias

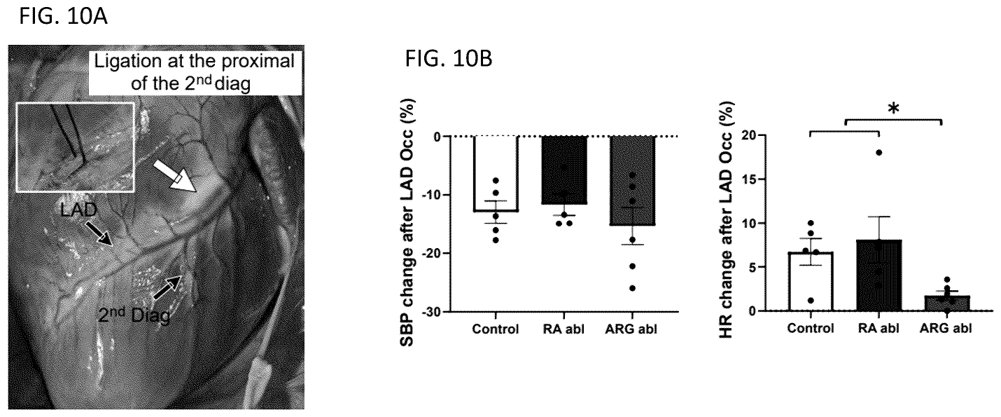

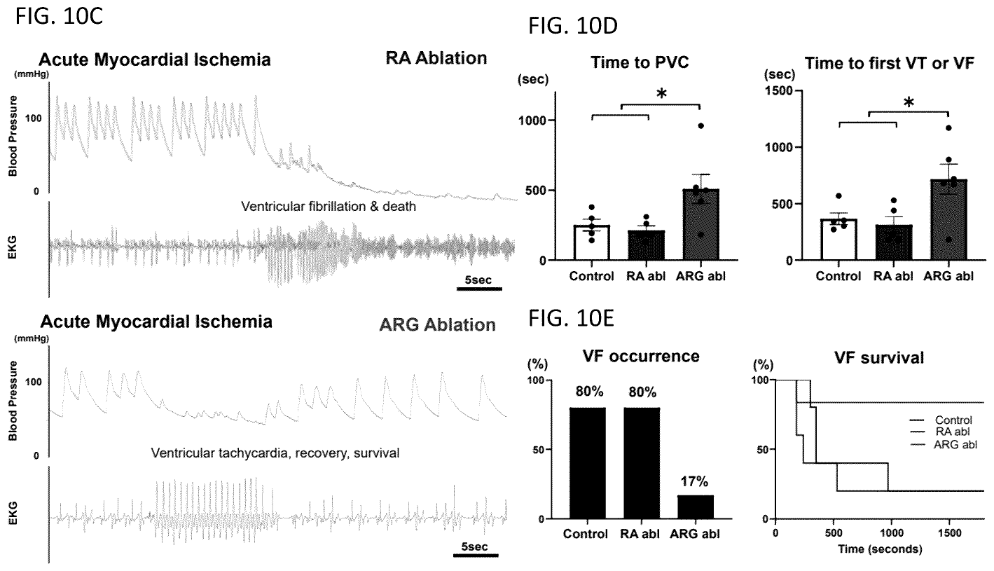

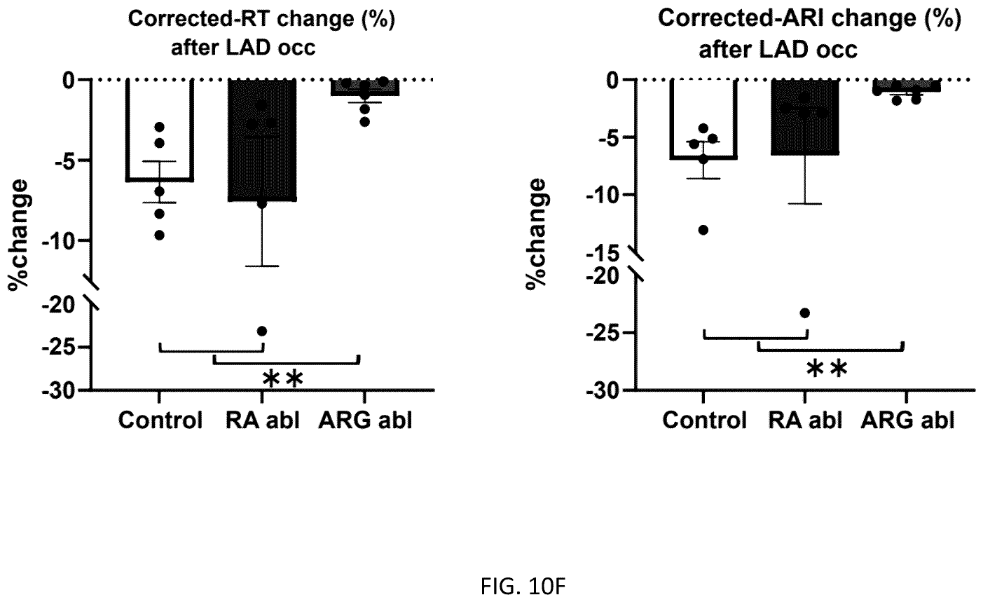

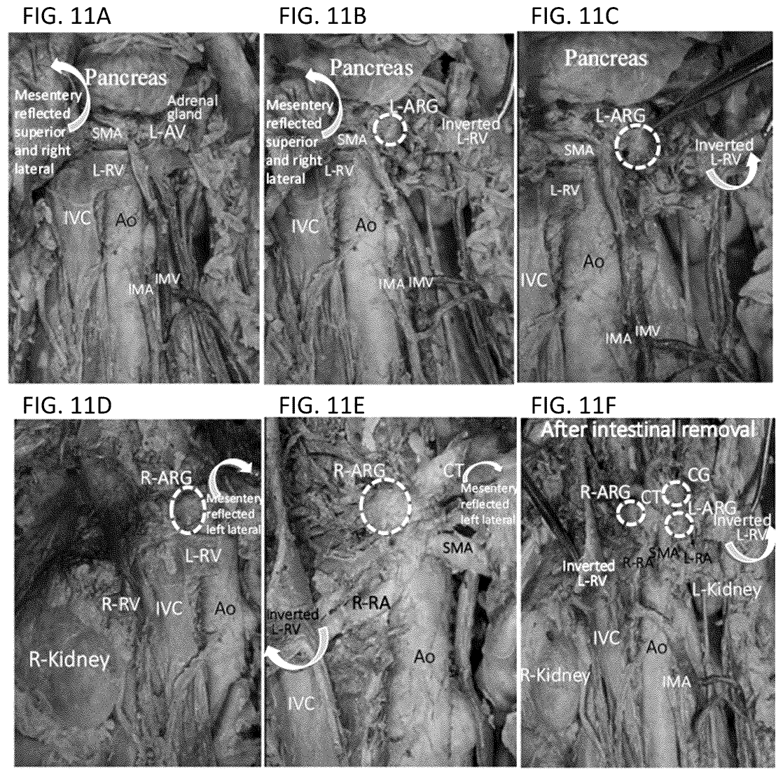

The assessment of whether greater renal denervation associated with ARG compared to RA ablation offers greater protection against acute ischemic ventricular arrhythmias compared to RA ablation and Control was performed. LAD occlusion was performed as shown in FIG. 10A in Control, RA, and ARG ablation animals. Interestingly, while hypotensive response to LAD occlusion was similar across groups, subjects that underwent ARG ablation demonstrated less heart rate response to LAD occlusion compared to RA ablation and Control animals (6.7±1.5 vs. 8.1±2.6 vs. 1.8±0.5%, p=0.04, respectively for control, RA and ARG ablation animals, FIG. 10B). Shown in FIG. 10C are representative traces showing blood pressure and electrocardiograms in both RA and ARG ablation during ischemia. The subject with ARG ablation had ischemic VT, but survived, whereas the RA ablation animal developed ventricular arrhythmias which degenerated into ventricular fibrillation (VF). In the ARG ablation group, the time to occurrence of PVC's (burden of 4:1 or more frequent) was longer compared to the Control and RA ablation groups (250±42 vs. 212±32 vs. 510±103 sec, p=0.02, respectively for Control, RA and ARG ablation subjects, FIG. 10D). The time to occurrence of VT or VF was also compared and found to be prolonged in ARG ablation cases (Time for first VT or VF in Control vs. RA ablation vs. ARG ablation; 366±53 vs. 312±73 vs. 718±133 sec, p=0.02, respectively, FIG. 10D). VF occurrence was 17% in ARG ablation, while Control and RA ablation were found to have occurrences of 80% (FIG. 10E). The survival curves among three groups are shown in FIG. 10E (p=0.08).

To examine possible mechanisms for protection against arrhythmias, repolarization parameters were assessed in the ischemic border zone where electrograms did not have significant ST-segment elevation and local repolarization times (RT) could be determined. Corrected-RT and corrected-ARI during the LAD occlusion prolonged significantly in Control and RA ablation but not ARG ablation animals (−6.4±1.3%, −7.6±4.0% and −1.0±0.4%, respectively, p<0.01, for corrected-RT and −7.0±1.6 vs. −6.6±4.2 vs. −1.1±0.2 sec, p<0.001, for corrected-ARI, FIG. 10F).

### Human ARG Anatomy

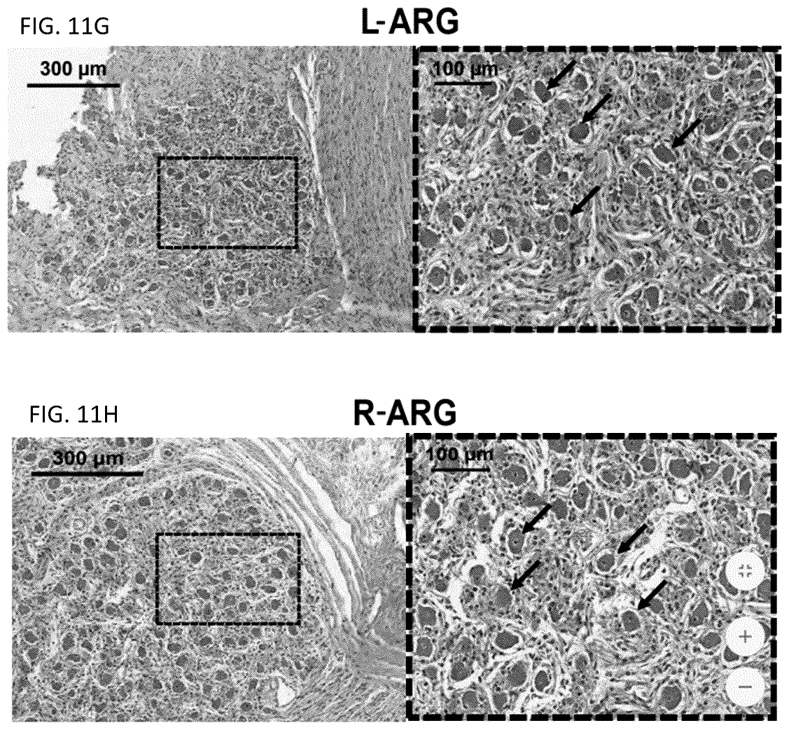

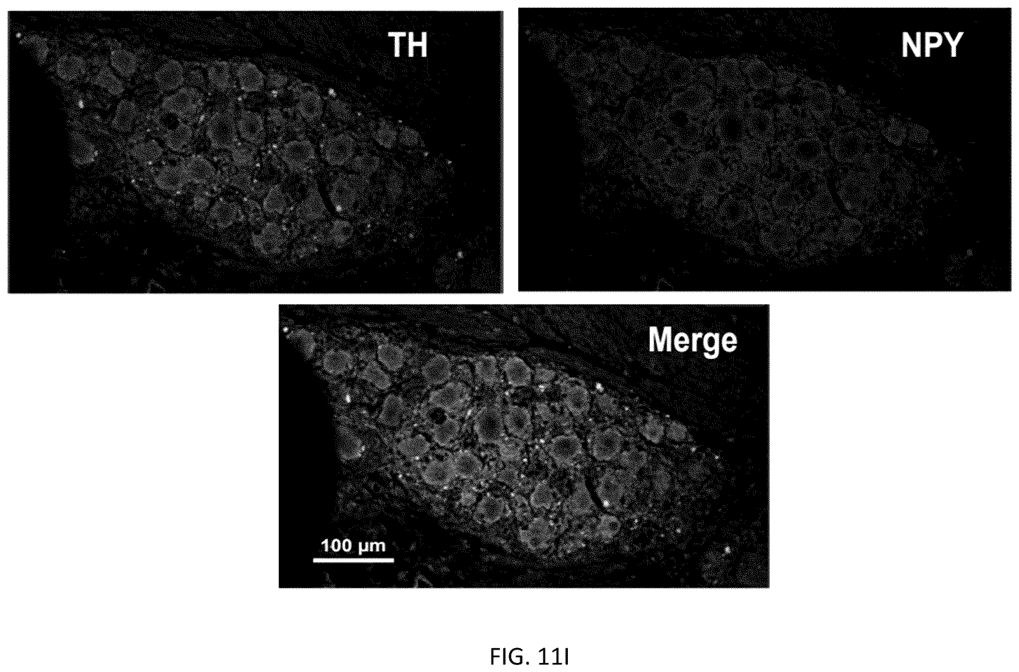

To explore translatability of these findings, the location of the ARG was studied in a cadaveric specimen. It was found that the ARG location is comparable to that seen in the swine. In humans, the L-ARG is observed next to the SMA, and posterior to the L-RV (FIG. 11A through FIG. 11C). The R-ARG can be located between the IVC and descending aorta, and superior to the R-RA (FIG. 11D, FIG. 11E). Both ARGs were located on the posterior side of the vein (FIG. 11F). The right and left ARGs consist of a large population of neurons while also containing nerve fibers that run in-between the observed neurons, similar to that of the swine model in the present study. These groups of neurons and their corresponding nerve fibers were visualized in H&E staining (FIG. 11G, FIG. 11H). Further IHC analysis of the ARG's with TH and NPY showed these neurons to consist primarily of adrenergic neurons (FIG. 11I), confirming the ARGs in humans to also be sympathetic neurons.

The main findings are; 1) Hemodynamic responses to ARG and RA stimulation are substantially influenced by the anesthetic regimen used; 2) hemodynamic responses to ARG stimulation are greater that than seen for RA stimulation; 3) RA ablation eliminated responses to RA stimulation only, while ARG ablation eliminated both ARG and RA stimulation responses; and 4) ARG ablation was protective against ischemic-induced ventricular arrhythmias and sudden death compared to RA ablation and control (i.e. sham/no ablation). This study is the first to compare the effects of ARG and RA stimulation and ablation in porcine, including how ablation impacts ischemia-induced ventricular arrhythmia and sudden death.

### ARG as the Novel Target for Renal Denervation

The present study showed that ARG ablation completely suppressed the effect of not only ARG stimulation but also that of RA stimulation. This suggests that the ARG has a potential role in RND. A previous study reported that the ARG was closely related with the renal nerve, both structurally and functionally (Norvell J E, Journal of Comparative Neurology. 1968 May; 133 (1): 101-11). Qian et al. also recently reported that the ARG was a potential target for RND in sheep (Qian P C et al., JACC: Cardiovascular Interventions. 2019 Jun. 24; 12 (12): 1109-20). However, they also discussed that ARG ablation would not completely denervate the ipsilateral kidney because there were variable amounts of adipose tissue surrounding the ARG. This is an important point when considering the ARG as a therapeutic target for RND. In the present study, however, both left and right ARGs can be detected effortlessly, regardless of surrounding adipose tissue and swine anatomical differences (FIG. 2A through FIG. 2I). This was aided by an anesthetic regiment that did not suppress autonomic reflex-mediated blood pressure responses (alpha-chloralose). Furthermore, both ARGs are located on the posterior side of the vein in swine. Thus, ablation was easily accomplished via the vein (mean total ablated time was 690±108 seconds). It could be deduced that ARG can be a sufficient target for RND, but there might be species-specific differences related to as the ARG location varies in each species (Norvell J E, Journal of Comparative Neurology. 1968 May; 133 (1): 101-11; Mizeres N J, American journal of Anatomy. 1955 Mar; 96 (2): 285-318).

The complex neuroanatomy surrounding the ARGs should be highlighted. ARGs exchange fibers with the celiac ganglion and at times may be fused with it. Fibers may also transit through the ARG to the superior mesenteric ganglion (SMG), although primary inputs to the SMG originate lower (T12 to L1) compared to the ARG's inputs which originate primarily from T10-T11. Care should also be taken to avoid fibers that run along the gonadal arteries to the testicles and ovaries, although these originate in preaortic ganglia below bilateral renal arteries. Importantly, nerve supply to the ampullae, seminal vesicles, and prostate originate well below the ARGs at the bifurcation of the aorta, substantially minimizing the risk of sexual side effects if ARG ablation were extended to humans. Interconnections to other plexi e.g. intermesenteric, celiac, and adrenal suggest that these intra-abdominal organs may be impacted by stimulation or ablation of the ARG. These may be mitigated by the fact that primary nerve supply to these organs via the celiac, mesenteric, and other ganglia are unlikely to be directly impacted by careful ARG ablation. Additionally, other forms of neuromodulation applied to the ARG (e.g. ultrasound) may avoid this altogether.

### ARG Ablation and Antiarrhythmic Effect

In the present study, the contribution of ARG ablation on the suppression of ischemia-induced ventricular arrhythmias was investigated. It is known that ventricular arrhythmias are related to sympathetic activity. Not only does ischemia activate the cardiac sympathetic nervous system, but sympathoexcitation results in the ventricular arrhythmias by various mechanisms including systemic release of catecholamines and altered cardiac electrophysiology. The present study found ARG stimulation had a significant impact on stellate ganglion activity, catecholamine release, and hemodynamic response (FIG. 5A through FIG. 5G) with ARG ablation eliminating the subsequent ARG stimulation responses (FIG. 7A through FIG. 7G). A previous study also reported the ARG ablation reduced the ipsilateral renal cortical norepinephrine content (Qian P C et al., JACC: Cardiovascular Interventions. 2019 Jun. 24; 12 (12): 1109-20). This explains the finding that ARG ablation offers greater protection against acute ischemic-induced ventricular arrhythmias and death compared to RA ablation and Control. ARG stimulation compared to that in RA stimulation was shown to have longer and more variable ARIs which is known to cause arrhythmias (FIG. 5D, FIG. 5E). The fact that ARG ablation prevented sudden death and offered ischemic-induced ventricular arrhythmia protection despite this hyper-stimulated state points to the potential of ARG ablation.

### ARG Stimulation Compared to RA Stimulation

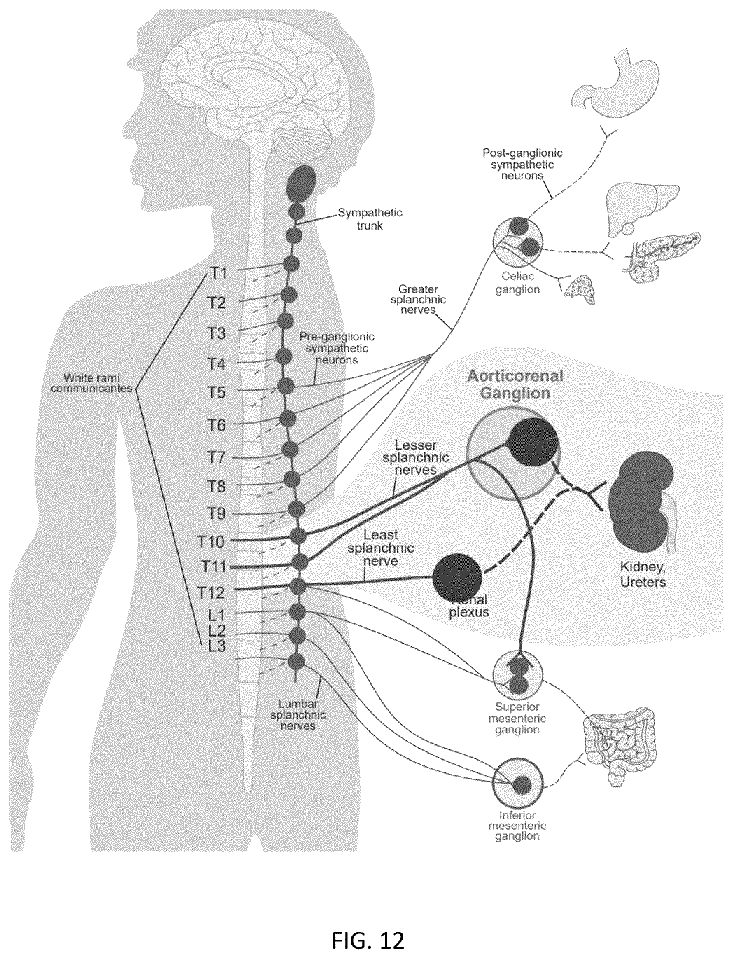

The observation of larger hemodynamic responses and persistently enhanced SG activity in response to ARG vs. RA stimulation demonstrates increased cardiac sympathetic outflow during ARG stimulation (FIG. 5A through FIG. 5G). This may be explained by two potential mechanisms. One is the different sensitivity in response to the stimulation. Sun et al., had a much greater increase of BP during ARG stimulation than RA stimulation, potentially due to the increased sensitivity of the ARG to stimulation (Sun J et al., Pacing and Clinical Electrophysiology. 2015 Jul; 38 (7): 825-30). The other explanation is the anatomical relationship with ARG and renal nerves around the RA. The ARG is a sympathetic ganglion, positioned upstream of the renal nerves, in which pre and post-ganglionic sympathetic nerves gather (FIG. 12). Thus, ARG stimulation would capture the target neurons more specifically and directly than RA stimulation. RA stimulation would have variously different responses from ARG stimulation because it is unable to capture all renal nerves surrounding the RA. A previous study also reported that RA stimulation had both weak and strong response sites due to variance in nerve distribution (Liu H et al., Hypertension. 2019 Sep; 74 (3): 536-45).

### Clinical Implications

The modulation of SG, including bilateral cardiac sympathetic denervation (BCSD) is promising in the management of ventricular arrhythmias (Dusi V et al., Heart Rhythm. 2020 Apr. 20; Ajijola O A et al., Journal of the American College of Cardiology. 2012 Jan. 3; 59 (1): 91; Meng L et al., JACC: Clinical Electrophysiology. 2017 Sep. 18; 3 (9): 942-9; Fudim M et al., Journal of cardiovascular electrophysiology. 2017 Dec; 28 (12): 1460-7). Moreover, it might be effective treatment for heart failure patients (Conceição-Souza G E et al., European journal of heart failure. 2012 Dec; 14 (12): 1366-73). However, not all patients can receive BCSD, because it is an invasive therapy. The present study highlights the relationship between ARG activation and SG firing. Since ARG ablation protects against ischemia-induced ventricular arrhythmias, and given its minimally invasive nature, transvenous ARG catheter ablation may have a role in reducing sympathetic flow and could ultimately offer protection from ventricular arrhythmias in patients who are not candidates for BCSD, or as an adjunct to it. Indeed, small initial studies of renal denervation after BCSD (Bradfield J S et al., Heart Rhythm. 2020 Feb. 1; 17 (2): 220-7) have shown promising results underscoring this concept.

### Example 2: Novel Approach to Modulate Renal Neurotransmission Via Percutaneous Approaches

Renal denervation to modulate ventricular arrhythmia continues to show promise. This suggests that the variability in efficacy relate to factors that could be overcome to improve the applicability and success of this therapy. The following studies were aimed at better understanding structural and functional renal innervation such that renal denervation in treating cardiovascular diseases.

### Anesthetic Approach

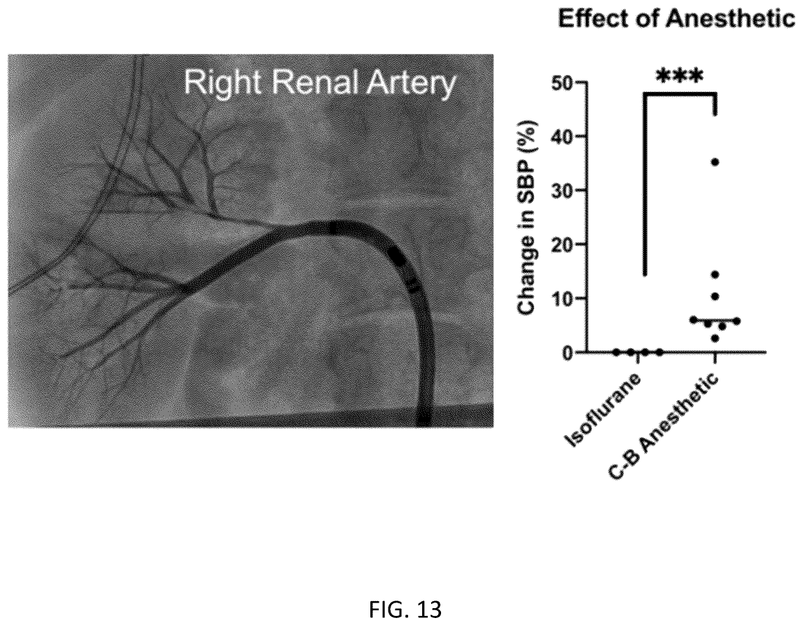

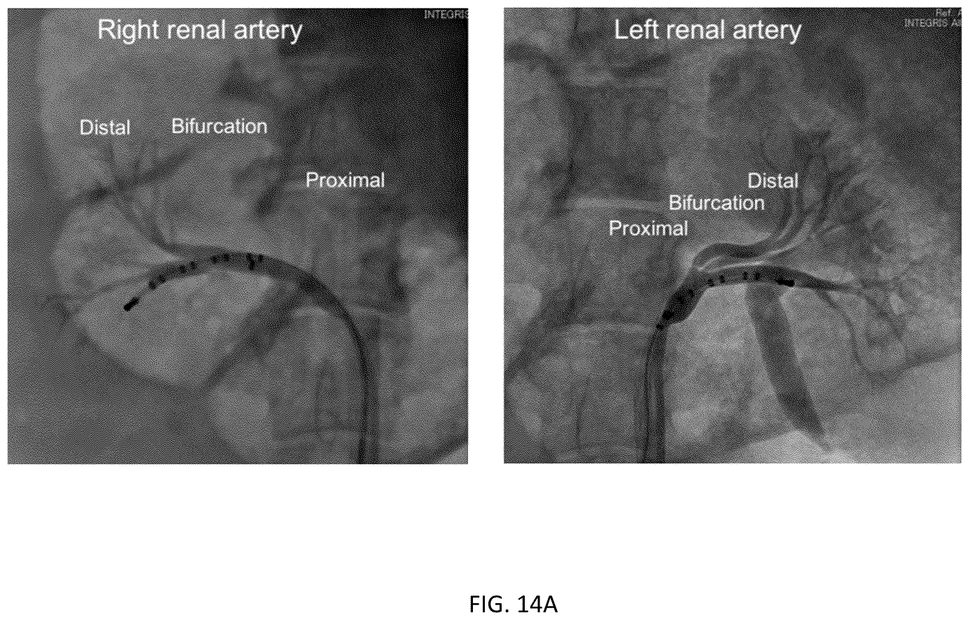

Anesthetic used has a substantial effect on the ability to generate robust hemodynamic responses. As shown in the FIG. 13, when isoflurane 1-2% was used, it blunted the blood pressure and heart rate responses to adequate stimulation parameters, however, when a cholarolose-based anesthetic agent was used, this yielded robust hemodynamic responses. Accordingly, sites that create hemodynamic responses can be consistently identified, such that those sites can be ablated to eliminate renal neurotransmission.

### Stimulation and Ablation Site

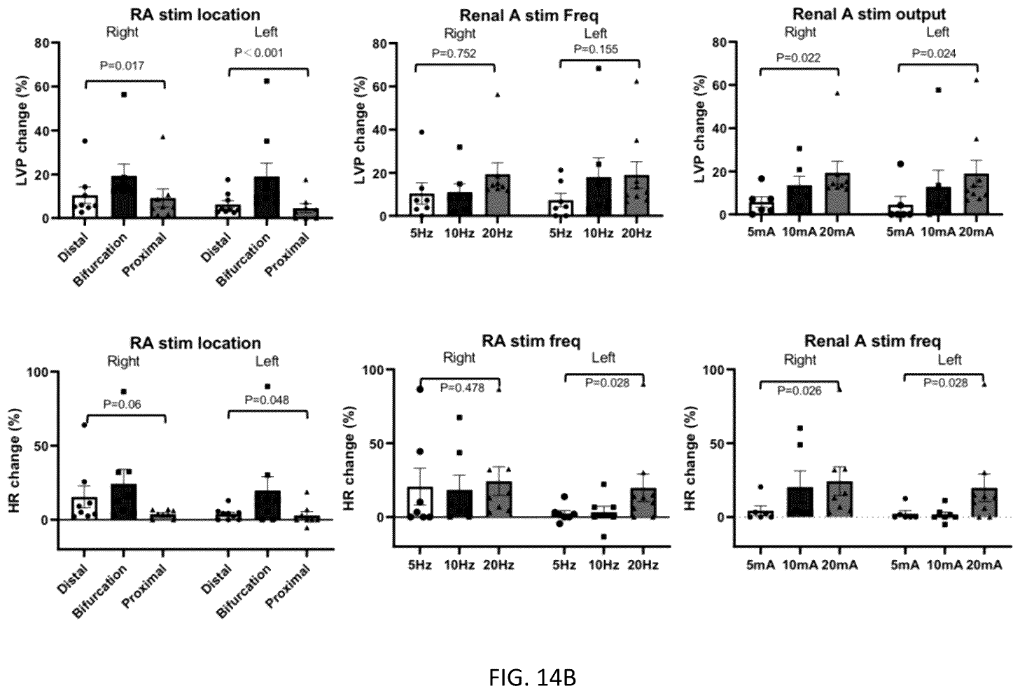

Stimulation parameters were examined using an optimal anesthetic regimen for reliable renal artery stimulation. The renal arteries were also mapped to better define anatomy models and to identify optimal stimulation sites. The responses at the sites were defined by examining various combinations of stimulation amplitude, frequency, and site. These parameters were examined in both kidneys to exclude the effect of anatomic differences based on laterality. FIG. 14A shows examples of site demarcation, while FIG. 14B shows the effects of different stimulation approaches.

As reflected in the graphs in FIG. 14B, the most potent responses for renal artery stimulation were found at the bifurcation of the renal artery on both sides. While 10 Hz and 20 Hz stimulation frequency yielded robust responses, overall 20 Hz yielded responses at more sites than did 10 Hz. Finally, 20 mA stimulation amplitude produced the best responses.

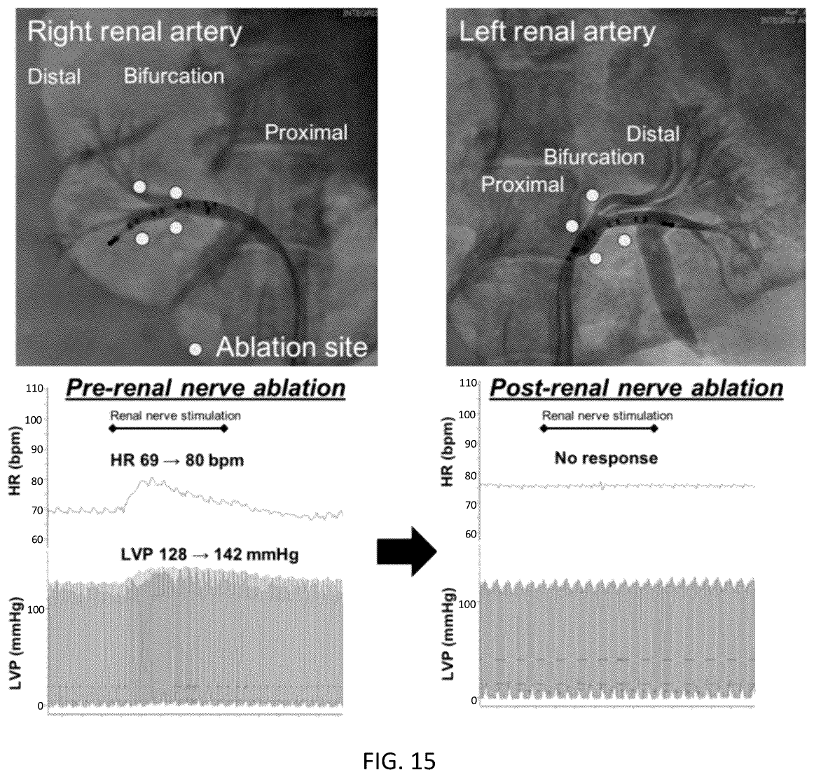

In summary, stimulation using 20 Hz, 20 mA for 60 seconds at the renal artery bifurcation yielded the best responses and led to excellent ablation results when stimulated at these sites as shown in FIG. 15. Renal denervation was performed by ablating proximal and distal of the renal artery bifurcation for a total of 8 points using ablation parameters of 20 W, 30 seconds, and flow of 8 mL/sec.

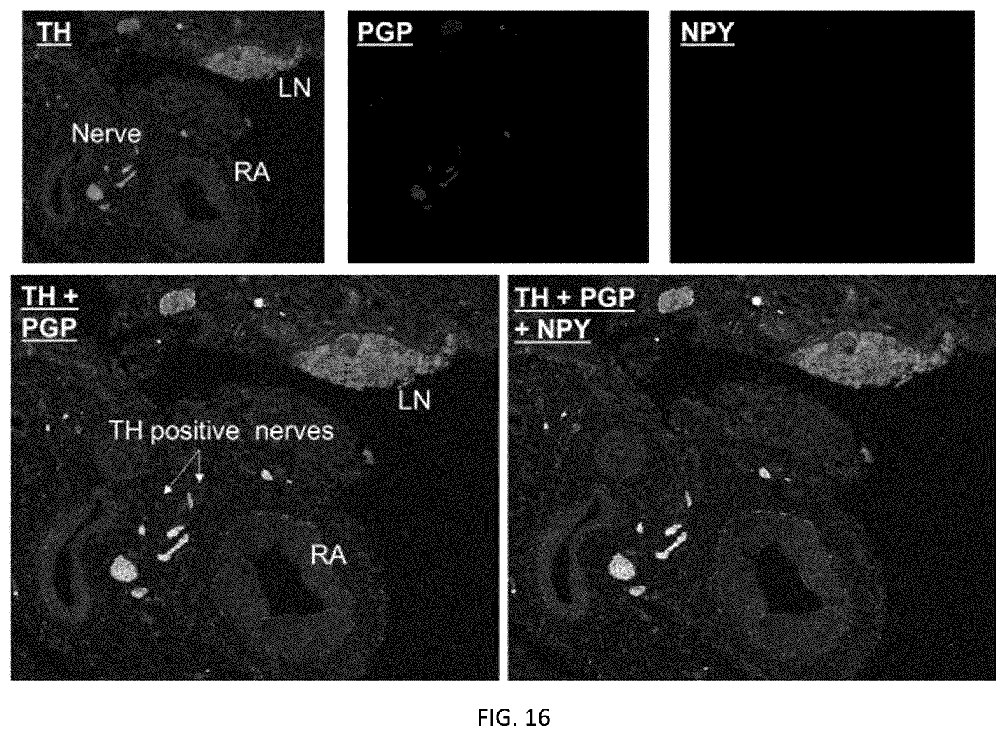

Immunohistochemistry was performed at the bifurcation and several large nerve bundles and fibers were identified (FIG. 16). These fibers were predominantly adrenergic. Staining was performed for sensory fibers (which identify afferent nerves), and such fibers were similarly identified at the bifurcation (not shown).

### Identification of Novel Renal Artery Ablation Sites

An important aim of this study was to identify whether novel renal neuromodulation sites could be utilized to denervate the kidneys. To this end, the anatomy of renal innervation was reviewed, with particular examination of sites that could be targeted endocardially. A site known as the aorticorenal ganglion (ARG) was found, which can be targeted endovascularly via the inferior vena cava (IVC) and renal venous system.

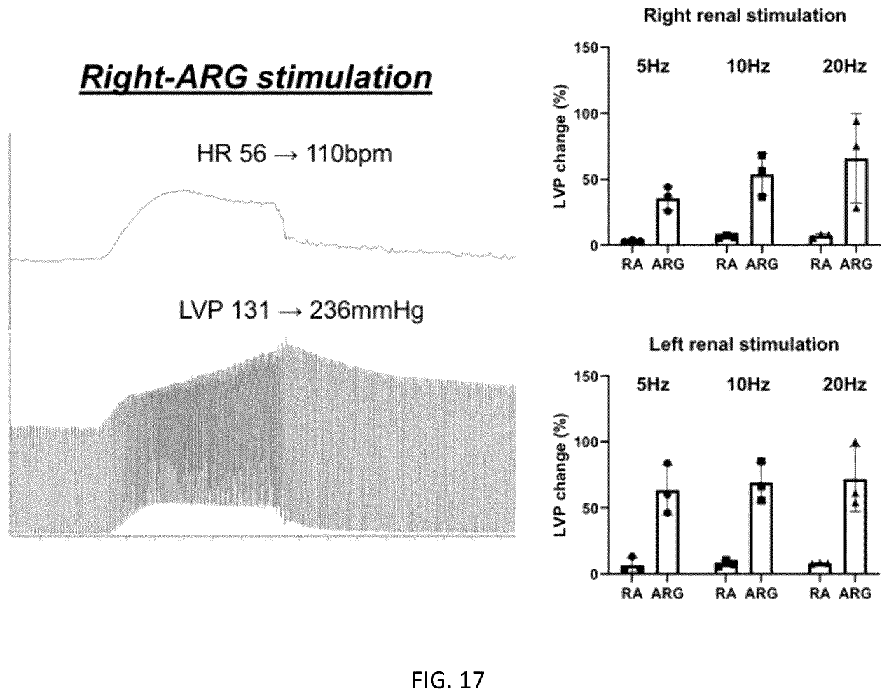

Stimulation at this site was compared to renal artery to verify that the ARG was an important site for renal blood pressure regulation by comparing the hemodynamic response at the ARG with that at the renal artery. As shown in FIG. 17, stimulation at 20 mA for 60 seconds at the ARG yielded a dramatic blood pressure response. As shown in the representative example in FIG. 17, systolic blood pressure increased by 105 mmHg from baseline and heart rate doubled. In comparing ARG stimulation to renal artery stimulation, the differences were substantial.

This degree of response demonstrates that the ARG is a site from which the renal artery nerves originate. Additionally, a known phenomenon in neuroscience is that ablation of axons does not yield as durable a response as the ganglia where the neurons from which these axon arise. In summary ARG yields a more robust and durable denervation at the kidneys.

### Characterization of Endovascular Approaches to the ARG

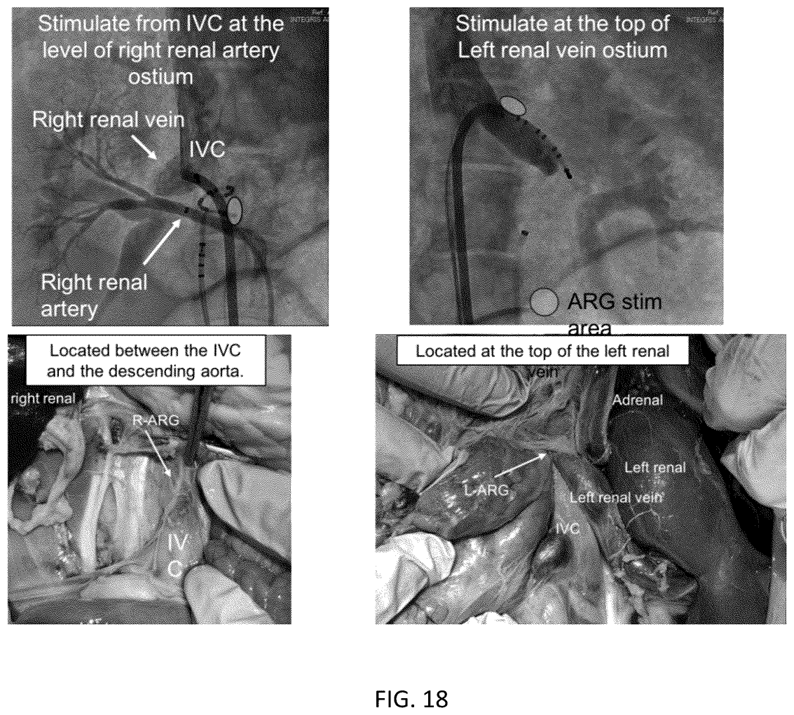

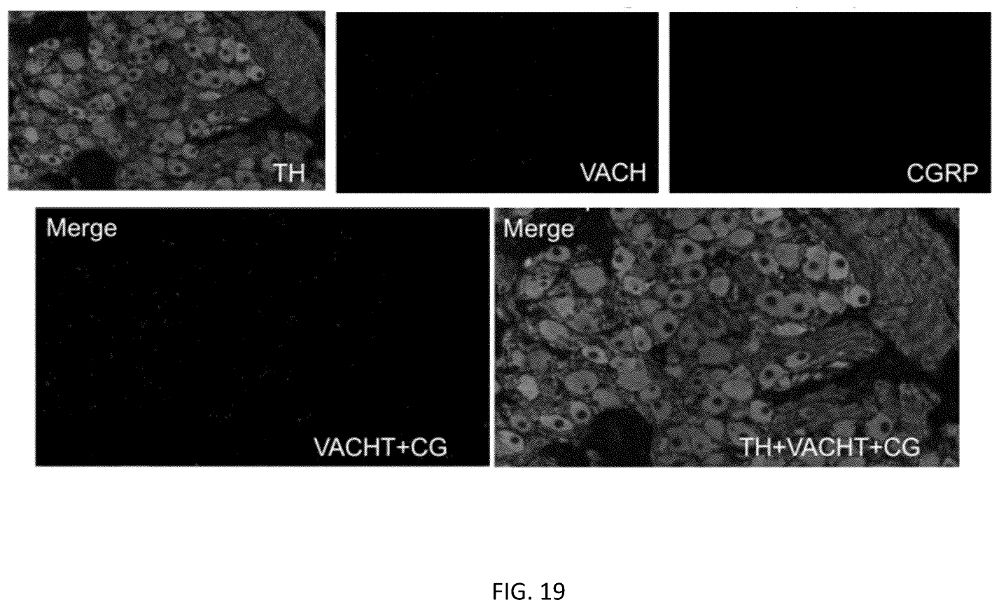

A series of studies were performed to confirm that endovascular approaches target the ARG, wherein fluoroscopy-identified endovascular sites were matched with gross anatomy as shown in FIG. 18. The site identified as the ARG was then verified to contain neural structures (specifically neuronal soma, and that some are adrenergic in nature). As shown in FIG. 19, ARG was identified to contain post-ganglionic adrenergic neurons based on extensive immunohistochemical stainings.

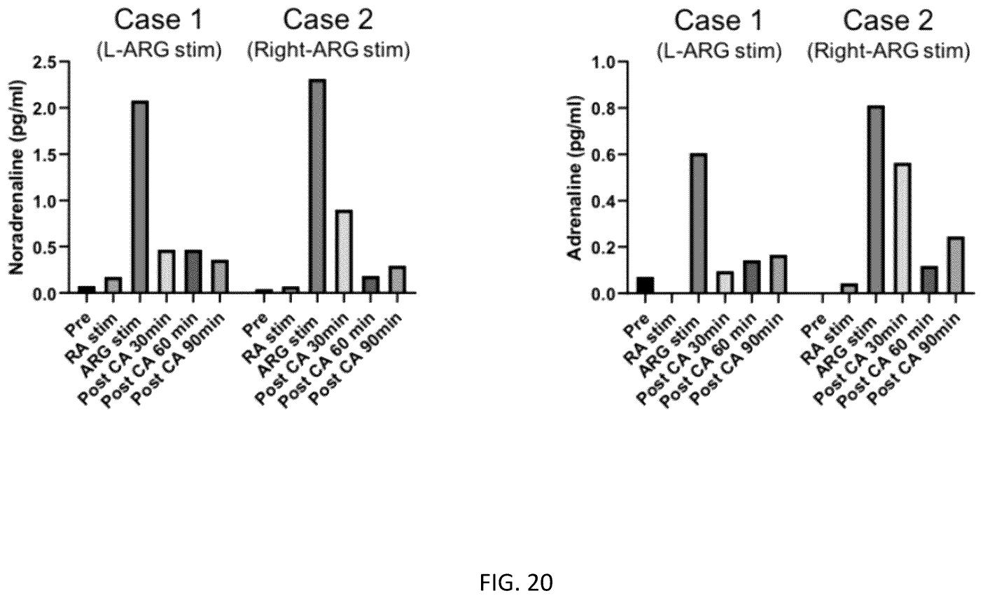

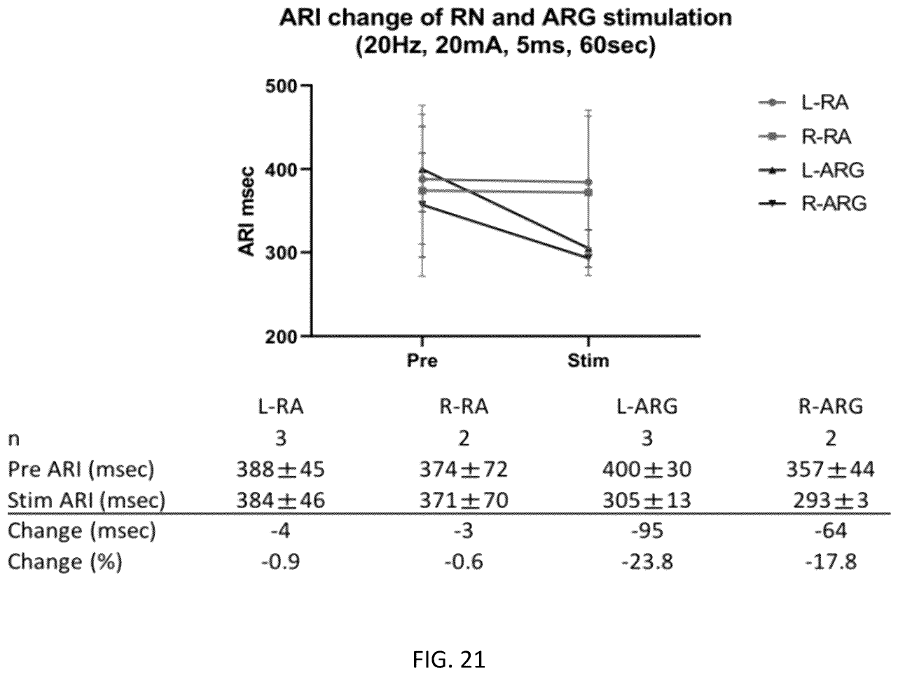

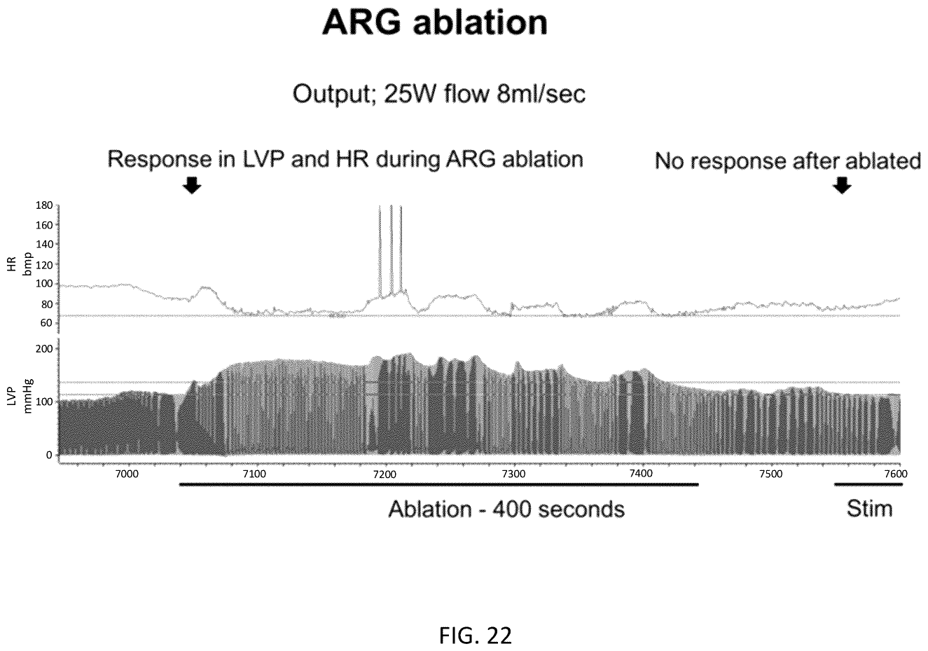

Given the massive pressure and heart rate response to ARG stimulation, ARG stimulation was confirmed to cause greater norepinephrine release than renal artery stimulation. Enzyme-linked immunosorbent assays were performed for noradrenaline and adrenaline for the following conditions: Baseline (Pre); renal artery (RA), Aorticorenal ganglion (ARG), coronary artery occlusion (CA). As shown in FIG. 20, ARG stimulation caused more catecholamine release than RA stimulation. Further, ARG stimulation cause greater catecholamine release than coronary artery occlusion. These findings demonstrate that the ARG is a major source of acute catecholamine release such that many physiological and pathophysiological processes are impacted by the ARG.

To further examine this, the effect of ARG activation on cardiac electrophysiology was investigated. Corrected activation recovery intervals (cARI) were measured, which is a measure of action potential duration (APD) in live beating hearts (independent of heart rate). As shown in FIG. 2I, RA stimulation did little to modulate cardiac ventricular APD; however, ARG stimulation strongly modulated cardiac electrophysiology. Both left and right ARG stimulation (blue markers and lines) significantly shortened myocardial APD compared to RA stimulation (red markers and lines). These data demonstrate that via massive systemic catecholamine release, ARG stimulation exerts more potent control of ventricular EP than renal artery.

The ARG can be effectively ablated, as shown in FIG. 22. Interestingly, during ablation, there is activation of the ARG, and this itself can be used as a marker of the appropriate site. Additionally, a linear ablation of 25 W, flow of 8 mL/sec is typically required to ablate the ARG. Upon completion of the linear ablation (approximately 400 seconds), no further activation of the ARG can be achieved by stimulation at 20 Hz, 20 mA, 60 seconds.

In summary, the present study demonstrated that renal nerve activation is facilitated by optimal anesthetic regimens. An optimal renal artery stimulation parameter is 20 Hz, 20 mA for 60 s at the renal artery bifurcation. The Aorticorenal ganglion (ARG) is an important target for renal neuromodulation as it: appears to be a purely adrenergic ganglion, consisting of post-ganglionic adrenergic soma to the kidneys; causes substantially larger hemodynamic responses to stimulation compared to renal artery stimulation; causes much greater norepinephrine and epinephrine release compared to renal artery stimulation; is a much stronger modulator of ventricular cardiomyocyte electrophysiology compared to renal artery stimulation; can be successfully targeted and ablated endovascularly, resulting in inability to activated renal nerves following ablation; and eclipses other studies in human and animal models in terms of hemodynamic responses to stimulation

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of renal denervation, comprising the step of administering ablation therapy to an aorticorenal ganglion (ARG).

2. The method of claim 1, wherein the ablation therapy is performed endovascularly through the inferior vena cava and renal vein.

3. The method of claim 1, wherein the ARG is selected from a left ARG, a right ARG, or both.

4. The method of claim 3, wherein ablation therapy is applied to the left ARG at a location positioned adjacent to a superior mesenteric ganglion and on a posterior side of a left renal vein.

5. The method of claim 3, wherein ablation therapy is applied to the right ARG at a location positioned between an inferior vena cava and descending aorta, superior to a right renal artery, on a posterior side of a right renal vein.

6. The method of claim 1, wherein the ablation therapy is selected from the group consisting of: cryoablation, radiofrequency ablation, chemical ablation, and laser ablation.

7. The method of claim 6, wherein the ablation therapy is radiofrequency ablation at 20 W using an irrigated catheter with a flow rate of 8 mL/second, for at least 30 seconds.

8. The method of claim 1, wherein the method further comprises a step of recording a baseline hemodynamic response before the step of administering ablation therapy.

9. The method of claim 8, wherein the step of administering ablation therapy generates an increase in the hemodynamic response.

10. The method of claim 9, wherein the method further comprises a step of ceasing ablation therapy once the hemodynamic response returns to baseline.

11. The method of claim 8, wherein the hemodynamic response is heart rate, blood pressure, or both.

12. The method of claim 1, wherein the method is effective in treating systemic adrenergic activation.

13. The method of claim 1, wherein the method is effective in treating a cardiovascular disease or disorder.

14. The method of claim 13, wherein the cardiovascular disease or disorder is selected from the group consisting of: atrial fibrillation (AF), ventricular arrhythmia, ventricular tachycardia, systolic heart failure (reduced ejection heart failure), diastolic heart failure (preserved ejection heart failure), myocardial infarction, hypertrophy, and hypertension.

15. The method of claim 1, wherein the ablation therapy suppresses sympathetic nerve activation.

16. The method of claim 1, wherein the ablation therapy suppresses effect of renal artery stimulation.

17. The method of claim 1, wherein the method is configured to modulate ventricular arrhythmia.
